(19) United States
(12) Patent Application Publication
(10) Pub. No.: US 2002/0172937 A1
(43) Pub. Date: Nov. 21, 2002

(54) Rapid assay for arthopod-borne disease vectors and pathogens

(76) Inventors: Kirti Dave, Thousand Oaks, CA (US); Eva Emmerich, Oxnard, CA (US); Jeffrey Ryan, Haymarket, VA (US); Robert A. Wirtz, Atlanta, GA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/505,898

(22) Filed: Feb. 17, 2000

Related U.S. Application Data
(60) Provisional application No. 60/120,872, filed on Feb. 19, 1999.

(51) Int. Cl. C12Q 1/70; C12Q 1/68; A61K 31/70; A01N 43/04; C12P 21/06; C12P 19/34

(52) U.S. Cl. 435/5; 435/6; 435/91.1; 435/69.1; 435/91.2; 514/44

(57) ABSTRACT

A method is described for detecting arthropod-borne human pathogens in arthropods using a rapid dipstick format where the signal for one or more pathogens can be visually detected on a single test dipstick. Specific examples of pathogens detected include Plasmodium species, togaviruses, flaviviruses, encephalitis viruses and Ross River virus. These tests are rapid, easy to use and are suited for use in the field.

This invention was made in part with support from grant number DAMD17-97-c-7020.

### FIELD OF THE INVENTION

The present invention is directed to rapid assays for detecting disease vectors and pathogens carried by arthropods and kits for carrying out the assays.

### BACKGROUND OF THE INVENTION

Arthropod-borne infections, including those such as malaria which are transmitted by mosquitoes, are often serious resulting in significant morbidity and even death. Figures from the World Health Organization in the 1996 World Health Report show that 2.1 million deaths were caused by malaria alone in 1995 (Day, “Scourge of infections Kills Third World's Young” in New Scientist, 150(2031): p6, 1996).

A protozoan of the genus Plasmodium, which includes P. falciparum, P. vivax, P. ovate and P. malariae, causes malaria in humans. P. falciparum, which can result in a potentially fatal infection, is widespread throughout the tropics and therefore constitutes an important health threat for millions of people. P. vivax is also widespread, and because of its propensity for successive relapse from liver and consequent toxicity, constitutes an important cause of morbidity in tropical regions. P. ovale and P. malariae are less common, both causing low-grade, chronic diseases, the latter infection often causing disruption of kidney function through immune-complex deposition.

Accurate methods for detecting malaria and other arthropod-borne infections are necessary to identify infected individuals so as to properly direct therapy and for identifying further sources that may increase disease spread. The standard and most cost-effective method for detecting malaria pathogens in mosquitoes involves isolation of sporozoites from salivary glands and enumeration using phase microscopy on a hemocytometer. However, the method is not very specific, is very labor intensive, and requires an effective microscope and skilled technicians. This method is not generally feasible in many regions of the tropics.

A recent resurgence of malaria in most endemic areas has stimulated the search for new methods to measure and control transmission. The absence of an effective vaccine, vector resistance to insecticides, and development of drug resistance have contributed to the defeat of control measures. Thus, the availability of rapid, simple, sensitive and specific diagnostic tools is of prime importance in the suppression of malaria. This situation also applies to other arthropod-borne diseases including dengue, encephalitis and Ross River virus.

Malaria and other arthropod borne infections have been detected by indirect immunofluorescent antibody tests (Ramsey et al., Am. J. Trop. Med. Hyg., 35(2), pp. 234-238, 1986). Antibody tests include a number of immunoradiometric and enzyme-linked immunosorbent assays (ELISAs) that have been developed for testing Plasmodium sporozoites in mosquitoes (Zavala et al., Nature, 299:737-738 (1982); Burkot et al., Am. J. Trop. Med. Hyg., 33:227-231 (1984); Burkot et al., Am. J. Trop. Med. Hyg., 33(5), pp. 783-788, 1984; Zavala et al., Exp. Med., 157(1983): 1947-1957; and Collins et al., Am. J. Trop. Med. Hyg., 33(1984): 538-543. Oprandy et al. (J. of Clin. Microbiology, 28(8), pp. 1701-1703, 1990) have provided a method for processing and microfiltration of mosquitoes for malaria antigen detection in a rapid dot immunobinding assay.

Presently, most immunological based tests are designed for detection of infection in humans rather than detection of infection in arthropods, which are vectors of the disease. In addition, most immunological ELISA based methods require sophisticated and fragile equipment, making them impractical for field use. Because of these limitations, diagnosis of malaria and other arthropod-borne diseases in the field is based on symptomatology, whose intrinsic inaccuracy results in the significant overuse of drugs and potential acceleration of the development of resistance factors. Thus a need exists for a rapid, easy-to-use assay for detecting arthropod-borne diseases and pathogens in arthropod samples and which can be used in the field without specialized equipment.

### SUMMARY OF THE INVENTION

Accordingly, there has been provided, according to one aspect of the present invention, a method for analyzing an arthropod sample for the presence or absence of one or more analytes associated with the pathogen that causes human malaria, comprising:

a) contacting a liquid permeable support with the arthropod sample and one or more detectable analyte-specific reagents that bind specifically to a protein analyte associated with Plasmodium sporozoite, if present, to form analyte-reagent complexes, said support comprising at least one detection area, said area having an analyte-specific capture reagent immobilized therein, said capture reagent specific for the protein analyte associated with Plasmodium sporozoite, said capture reagent being adapted for capturing the analyte-reagent complexes; and

b) detecting the presence of the detectable analyte-specific reagent in the detection area, indicating the presence of the analyte in the sample.

In accordance with another embodiment of the present invention, the method employs at least two detectable analyte-specific reagents, said reagents specific for a protein associated with Plasmodium falciparum sporozoite and a second specific for a protein associated with a Plasmodium vivax sporozoite and at least two different detection areas, one area having immobilized therein a capture reagent specific for the protein associated with Plasmodium falciparum sporozoite, and a second area having immobilized therein a capture reagent specific for the protein associated with a Plasmodium vivax sporozoite.

In a further embodiment, the method can detect either Plasmodium vivax 210 or 247.

In another aspect of the present invention, a method is provided for analyzing an arthropod sample for the presence of at least one analyte associated with at least one type of arthropod-carried agent, wherein the arthropod-carried agent is a togavirus, comprising:

a) contacting a liquid permeable support with the arthropod sample and a detectable analyte-specific reagent that binds to an analyte associated with the togavirus, if present, to form analyte-reagent complex, said support comprising a detection area, said area having an analyte-specific capture reagent immobilized therein, said capture reagent specific for the analyte associated with the togavirus, said capture reagent being adapted for capturing the analyte-reagent complex; and

b) detecting the presence of the detectable analyte-specific reagent in the detection area, indicating the detection of the analyte in the sample.

In other embodiments, the method is designed to detect any of various arthropod-borne viruses including encephalitis viruses, dengue viruses and other Togaviridae viruses.

In yet another aspect of the present invention, a method is provided for analyzing an arthropod sample for the presence or absence of two or more analytes associated with an arthropod-carried agent, comprising:

a) contacting a liquid permeable support with the arthropod sample and at least two detectable analyte-specific reagents that bind to each of the analytes, if present, to form analyte-reagent complexes, said support comprising at least two detection areas, said areas each having an analyte-specific capture reagent immobilized therein, said capture reagent being adapted for capturing one of the analyte-reagent complexes; and

b) detecting the presence of the detectable analyte-specific reagent in each of the detection areas, indicating the presence of the analyte in the sample.

In further embodiments of these inventions, the analyte specific reagents are monoclonal antibodies or polyclonal antibodies that can be labeled with gold or colored latex.

In yet further embodiments, the sample is homogenized with a grinding solution prior to contact with said support.

In still yet further embodiments, the support further comprises a control area having immobilized therein at least one specific reagent for capturing detectable analyte-specific reagent.

In addition, the present invention provides kits for carrying out the above methods.

### BRIEF DESCRIPTION OF THE DRAWINGS

FIG. 1 is a photograph of dipstick panel assays for different arthropod-borne agents.

Panel A is a malaria panel assay for simultaneously detecting any of P. falciparum, P. vivax 210 or P. vivax 247 at a sensitivity of about 0.4 to 0.08 ng/ml antigen (see Examples 5 and 6 for assay details). With respect to capture antibodies printed on the membrane, the control line is at the top, Pf210 antibody is most proximal to the control line, Pv210 antibody is intermediate in position and the antibody Pv247 is most distal to the control line. Dipsticks were inserted into analyte control containing a mixture of Pf, Pv210 and Pv247 antigens, each at 12.5 ng/ml (lane 1), 4.2 ng/ml (lane 2), 0.8 ng/ml (lane 3), 0.4 ng/ml (lane 4), 0.2 ng/ml (lane 5), 0.08 ng/ml (lane 6) and 0 ng/ml (lane 7) (buffer only) in PBS with 0.5% NP-40.

Panel B is a dengue/Flavivirus panel assay for simultaneously detecting any of dengue virus serotypes 1-4 or any Flaviviruses at sensitivity of about a 1:2,000 dilution of antigen (see Example 7 for assay details). With respect to capture antibodies printed on the membrane, the control line is at the top, the one proximal to the control is monoclonal antibody 4G2 (flavivirus specific) and the one distal to the control is monoclonal antibody 2H2 (Dengue 1-4 specific). Dipsticks were inserted into analyte control solutions for Dengue 2 including 10× (lane 1), 100× (lane 2), 500× (lane 3), 1000× (lane 4), 2000× (lane 5) dilutions of Dengue 2 inactivated virus particles in PBS with 0.1% Tween-20 and a PBS detergent control solution (lane 6).

Panel C is an encephalitis panel assay for simultaneously detecting any of St. Louis encephalitis virus, Western equine encephalitis virus or Eastern equine encephalitis virus at a sensitivity of about a 1:2,000 dilution of antigen (see Example 8 for assay details). With respect to capture antibodies printed on the membrane, the control line is at the top, proximal to the control is monoclonal antibody 6B6C-1 (Flavivirus cross-reactive), most distal to the control is monoclonal antibody 1B5C-3 (EEE specific) and intermediate in position to the control is monoclonal antibody 2A3D-5 (WEE specific). Dipsticks were inserted into analyte control solutions containing a mixture of the encephalitis viruses (SLE, WEE, and EEE) at 10× (lane 1), 100× (lane 2), 500× (lane 3), 1,000× (lane 4), or 2,000× (lane 5) dilutions in PBS with 0.5% NP-40, and a PBS-detergent control solution (lane 6).

FIG. 2 depicts a scheme summarizing a Dipstick Malaria Sporozoite antigen panel assay. Processing of mosquitoes, detection of three different malaria species and interpretation of results is provided.

FIG. 3 shows a lateral flow plastic cassette containing a novel filter assembly (see Example 9 for more details).

FIG. 4 compares the sensitivity of CS ELISA versus dipstick assays for detection of Plasmodium falciparum antigen at the concentrations indicated. Details of the assay are described in Example 10.

FIG. 5 compares the sensitivity of CS ELISA versus dipstick assays for detection of Plasmodium vivax 210 antigen at the concentrations indicated. Details of the assay are described in Example 10.

FIG. 6 compares the sensitivity of CS ELISA versus dipstick assays for detection of Plasmodium vivax 247 antigen at the concentrations indicated. Details of the assay are described in Example 10.

FIG. 7 is a photograph of the a Plasmodium sporozoite panel assay performed on infected mosquitoes. Details of the assay are described in Example 10.

### DETAILED DESCRIPTION OF PREFERRED EMBODIMENTS

The present invention is directed to the diagnosis and control of human diseases transmitted to humans by contact with arthropods (i.e., vectors). Arthropods such as insects and ticks act as vectors of human disease when they become physically associated with the pathogen or biologically infected by the pathogen. The terms “arthropod-borne” and “arthropod-carried” agents are used interchangeably herein to refer to all agents that directly or indirectly cause disease in humans through direct or indirect contact with an arthropod which is physically associated with or biologically infected by the pathogen.

Provided herein are methods and kits for detecting arthropod-borne human diseases such as the parasites of malaria or viruses such as togaviruses, including encephalitis viruses, flaviviruses, dengue viruses, and Ross River viruses. The methods and kits of the present invention may be adapted for quantitative analysis as well as qualitative analysis.

As described herein, an arthropod sample obtained from the field is tested for arthropod-borne agents by detecting the presence of a specific analyte associated with the agent. “Arthropod sample” as used herein refers to a whole arthropod or multiple arthropods isolated from a natural population of arthropods, body parts of an arthropod (such as the head and thorax), homogenized arthropods, or any other arthropod form that permits detection of a desired analyte according to the present invention. The choice of arthropod depends on the infectious agent to be detected and the location where sampling is to take place.

Generally, the arthropod sample is treated with a liquid, such as an extraction solution or grinding solution, e.g. boiled casein (see Example 3), prior to testing. The arthropod sample may then be filtered to remove debris prior to testing. A preferred filter device is shown in FIG. 3.

As used herein, an “analyte associated with an arthropod-borne agent” is a molecule that is, or at one time was, physically associated with the agent and whose presence in the arthropod indicates infection or physical association of the agent with the arthropod. An arthropod-borne agent can be associated with at least one and generally several analytes, which are absent or different in other agents. An example of an analyte associated with an arthropod-borne agent is a Plasmodium circumsporozoite protein or epitopes of such protein.

In accordance with the method, the arthropod sample is contacted with a liquid permeable support and at least one detectable analyte-specific reagent that binds to the analyte. As used herein an “analyte specific reagent” is a molecule that can bind to an analyte associated with an agent. The analyte-specific reagent has been chosen such that under the conditions of use, it binds to a particular analyte associated with one or more agents, but not with other analytes of other agents. Thus, the analyte specific reagent can bind specifically with a particular analyte so that binding can be used to conclude (alone or in combination with other information) that the particular analyte associated agent is present in the arthropod sample.

Analyte specific reagents of the present invention include reagents that are well known in the art to exhibit binding specificity for an analyte associated with a pathogen. Such reagents are antibodies or other proteins that can provide binding specificity. As used herein, “antibody” includes, but is not limited to, any of a large number of proteins of high molecular weight that are produced normally by specialized B type lymphocytes after stimulation by an antigen and act specifically against the antigen in an immune response. Antibody typically consist of four subunits including two heavy chains and two light chains—also called immunoglobulin.

Antibodies also include naturally occurring antibodies as well as non-naturally occurring antibodies such as domain-deleted antibodies, Fab fragments, single chain Fv antibodies and the like. Monoclonal antibodies are the preferred analyte specific reagents. Methods to produce antibodies including polyclonal and monoclonal antibodies are well known in the art (see, e.g., Harlow and Lane, “Antibodies, a laboratory manual.” Cold Spring Harbor Laboratory, 1988). Non-naturally occurring antibodies can be constructed using solid phase peptide synthesis, can be produced recombinantly or can be obtained, for example, by screening combinatorial libraries consisting of variable heavy chains and variable light chains (e.g. see U.S. Pat. No. 5,969,108 to McCafferty).

The analyte-specific reagent of the present invention can be made detectable by physically or chemically attaching the reagent to a detectable moiety. A detectable analyte-specific reagent is preferably a colored analyte-specific reagent, wherein the color is visually identifiable, and more preferably, is a color having an intensity that can be seen with the unassisted human eye. Any color, including black and white, may be used. Preferable detectable moieties for analyte-specific reagents include a colloidal metal such as colloidal gold, carbon particle or a colored latex particle. Latex and carbon particle based assays are preferred when using densitometric-based readers for quantitative analysis. Suitable marker colors include dark blue and black latex as well as carbon particles.

As used herein, detectable analyte specific reagents also includes the term “conjugate” or antibody conjugate because the reagent can be chemically conjugated to the detectable moiety. The term “conjugate” is intended to include all types of chemical associations, whether they involve covalent or non-covalent forces. For example, colloidal-gold labeled antibody is an association based on non-covalent forces (i.e. adsorption) is considered a “conjugate” as this term is used herein.

Attaching a colored moiety to an analyte-specific compound such as an antibody is the preferred method of making the analyte-specific reagent. Color intensities that are not detectable to the human eye may be used and may be detected with the assistance of a color-detecting apparatus. In addition, other detectable analyte-specific reagents may be used that are known to those of ordinary skill in the art such as radiolabeled analyte-specific reagents. Other detection systems such as a magnetic moiety, an enzyme (in conjunction with a suitable substrate, the product of which is detectable), and the like also may be used. Accordingly, these alternative detection systems are within the scope of the present invention.

Immunochromatographic assays fall into two principal categories: “sandwich” and “competitive,” according to the nature of the antigen-antibody complex to be detected and the sequence of reactions required to produce that complex. In general, the sandwich immunochromatographic procedures call for mixing the sample that may contain the analyte to be assayed with antibodies to the analyte. The antibodies are mobile and typically are linked to a label or a disclosing agent, such as dyed latex or a colloidal metal sol such as gold. This mixture is then applied to a chromatographic medium containing a band or zone of immobilized antibody to the analyte of interest. When the complex of the antigen to be assayed and the labeled antibody reaches the zone of the immobilized antibodies on the chromatographic medium, binding occurs, and the bound labeled antibodies are localized at the zone. This indicates the presence of the antigen to be assayed.

Chromatographic medium in the form of a strip that runs vertically through the strip is often referred to as a “dip-stick” or “dipstick” whereas medium laid out in horizontal level are referred to a the “lateral-flow” formats. The lateral format have generally a plastic casing often called a “cassette” with a “sample well” where the sample is introduced onto the test strip.

In competitive immunoassays, the label is typically a labeled analyte or analyte analog which competes for binding of an antibody with any unlabeled analyte present in the sample. Competitive immunoassays are typically used for detection of analytes such as haptens, each hapten being monovalent and capable of binding only one antibody molecule.

As used herein, a “liquid permeable support” to which the arthropod sample and at least one detectable analyte-specific reagent is contacted, can be any type of material which is fixed in position and suitable for immobilization of a capture reagent while allowing the sample and analyte specific reagent (or complex of the two) to travel with the liquid phase through the support. The liquid phase moves through the support by capillary flow or wicking. The support preferably comprises cellulose, a derivative of cellulose, or a combination thereof and is in the shape of a rectangular strip, preferably, having a width of about 4 mm to about 5 mm. A preferred liquid permeable support is a dipstick which is well known in the art and readily available from commercial sources.

A typical dipstick consists of several overlapping and interconnected regions, which include a wick pad (referred to also as a sample pad), conjugate pad, porous chromatographic membrane and absorbent pad (referred to also as a reservoir), typically linked in this order. Optionally, a filter can be included to receive the sample which then passes to the wick pad. The wick pad consists generally of some amount of glass-fiber interwoven with cellulose. An example is glass fiber from Whatman (Grade:F075-17). Often this material is treated with polymers to prevent any non-specific binding of antigens of interest to the strip wick material (see, e.g., Jones, IVD Technology, vol. 5(no.2), p32, March/April 1999 or Jones, IVD Technology, vol. 3, (no. 3), p26, May/June 1999). Dimensions and nature of wick material also play an important role in the volume and hydrophobicity of the sample to be tested and the speed of development of results.

The conjugate pad can be coated with a wide variety of materials to provide for enhanced properties. Coatings (also referred to as protective agents) may include protein, polysaccharide, synthetic polymers, non-ionic detergents, sugars or the like, which are used particularly to enhance the stability of the detectable analyte specific reagent applied on the pad. Many specific examples of coating or protective agents are known in the art, including, gelatin, polyvinlypyrrolidone, casein, ovomucoid, polyvinylalcohol, polyacrolein, crystallized egg albumin, polyethyleneimine, potato starch, dextrin, polyethyleneglycol, NP-40, Tween-20 and Triton X-100. Such materials are generally applied to the pad prior to, or in combination, with the detectable analyte specific reagents The analyte specific reagent after application to the conjugate pad is then allowed to dry and can be stored at room temperature. The coating material and non-ionic detergents are important for “hydrating” the dried components once the liquid sample material comes in contact with the conjugate pad. In the P. falciparum assays, NP-40 detergent is preferred over other detergents.

The porous chromatographic membrane may be a nitrocellulose membrane or a nylon membrane or the like. Such porous membranes have the natural ability to bind proteins, and immunoreagents are applied directly to the membrane using specialized printing systems such as that from IVEK (North Springfield, VT; Biodot, Irvine, Calif.). The membranes are available in a broad range of pore sizes from about 1.0 micron to 15.0 micron. Generally membranes with pore size 5 to 12 microns are preferred.

The various regions of the dipstick are built over a support or “backing.” Backing material is generally non-porous, water insoluble, rigid and made of either polypropylene, polystyrene, polymethacrylate or nylon.

Distal to the wick and conjugate pad and at the other end of the membrane is the absorbent pad. The absorbent pad may be any hydrophilic material such as paper, sponge, or felt. The backing material as well as the absorbent pad material are preferably inert to any chemical reactions that occur on the membrane and such materials can contribute to “background” or “noise” on the membrane around the signal zone. Dipsticks and related lateral flow assays can be designed and manufactured by methods well known in the art (see, e.g., Carlberg, IVD Technology, vol. 5(no.3), p46, May/June 1999).

The overall size of the strip is dependent on several considerations. The primary consideration is to move a sufficient amount of analyte across to give a sufficient signal so that a sensitive and accurate assay is achieved. When capillary flow is predominantly upward, the length and thickness of the strip control the amount of solution that can pass along the strip. Generally a width of 4 to 5 mm and a length of 60 mm is a good approximation. The length of the strip will depend on the concentration of one or more of the analytes and practical considerations such as ease of handling.

In accordance with the present method and kits, the liquid permeable support comprises at least one detection area having an analyte-specific capture reagent immobilized therein, the capture reagent being adapted for capturing a complex (analyte-reagent complex) that forms when the analyte is bound by the analyte-specific reagent. The capture reagent, which is immobilized onto the detection area of the support, is preferably deposited as a stripe or a thin line perpendicular to the flow of liquid through the support. Once the capture reagent is deposited on the support, it is dried. Optionally, the detection area is overlaid with a blocking agent to immobilize it in place and/or to prevent non-specific binding by subsequent reagents (see, e.g., Jones, IVD Technology, vol. 5(no.2), p32, March/April 1999 or Jones, IVD Technology, vol. 3, (no. 3), p26, May/June 1999). Such blocking agents for immobilization are known to those of ordinary skill in the art, and include, for example, non-fat milk or bovine albumin.

The support preferably further includes a control area having immobilized therein for capturing the detectable analyte-specific reagents. In this manner, it is possible to ensure that the method or kit is working properly, since the excess unbound detectable analyte-specific reagents will be carried by the sample solution to the control area and be detected (e.g. a visible color). The reagent applied to the control area can be specific for the analyte specific reagent. For example, if the analyte specific reagent is a mouse monoclonal antibody, the control area can contain goat anti-mouse immunoglobulin antibody immobilized therein. Alternatively, the control area can have analyte or analytes immobilized therein.

Immobilization of analyte-specific reagents or analyte to the support can be performed by methods well known in the art. Such methods include nonspecific adsorption or chemical conjugation directly or through a spacer as is well known in the art (see. e.g. Hermanson, Bioconjugate Techniques, Academic Press, 1996; Harlow and Lane, supra). Chemical linkage also can be accomplished with homo or heterobifunctional cross-linking agents and the like such as are commercially available. The particular chemistry to be employed depends on the nature of the analyte and the support.

An analyte-specific capture reagent that is bound to the fluid permeable support can be the same or different from the analyte-specific reagent that is contacted with the arthropod sample. The detectable analyte-specific reagent must be compatible with the capture analyte-specific reagent such that the capture reagent can be adapted for capturing the analyte when bound to the detectable antibody specific reagent. The requirements to bind analyte in solution when the reagent is in solution versus attached to a solid phase are not identical and may depend on the binding affinity. It is important the analyte bound by the capture reagent be the same or physically associated with the analyte bound by the detectable reagent. For example, the two reagents can be specific for different epitopes or analytes present on the infectious agent.

Also, it is necessary to determine that binding of the detectable analyte specific reagent to the analyte does not hinder subsequent binding by the chosen capture reagent. For instance, in the case of monoclonal antibodies, the capture reagent and solution phase detectable reagent are usually chosen to react with different antigenic epitopes of an analyte. However, if a monoclonal antibody reacts with an repeated epitope of analyte, then a single antibody can provide both the detectable reagent in solution and the capture reagent on solid phase.

In accordance with the present methods, the arthropod sample is contacted with a liquid permeable support and at least one detectable analyte-specific reagent that binds to the analyte, the permeable support comprises at least one detectable area having a capture reagent immobilized thereto. The arthropod sample may be contacted first with the detectable reagent to allow the complex to form between analyte in the sample, if present, and the detectable analyte-specific reagent.

Alternatively, the detectable reagent may be contacted with the liquid permeable support before contacting the sample with the support. In this embodiment, the detectable analyte specific reagent can be added to a conjugate pad located adjacent to the test strip at an upstream sample entry location. The conjugate pad includes labeled antibody and any other assay reagents that may be desirable or required. In accordance with this embodiment, the end user simply adds an amount of the sample suspected of containing analyte to the conjugate pad, either directly or indirectly via a sample entry port. The sample entry point may optionally include a filter assembly such as is depicted in FIG. 3. The sample migrates through the conjugate pad, liberating and mixing sample fluid with the conjugate and any other assay reagents provided in the conjugate pad and the combined fluids migrate through the liquid permeable support including passage through the testing zone whereupon a signal may be developed. See, e.g., U.S. Pat. No. 5,075,078 to Osikowicz.

Without being bound by any theory, it is believed that the analyte-reagent complex travels with the liquid phase of the support by capillary action and accumulates at the site of the immobilized capture reagent when the later binds to analyte in the complex. Alternatively or in addition, unbound analyte may move with the liquid phase through the support and be retained at the site of immobilized capture antibody. The analyte thus bound can bind to detectable reagent moving with the liquid phase.

Preferably, each support contains a combination of gold-adsorbed and membrane-immobilized monoclonal and/or polyclonal antibodies to produce a distinctive visual pattern indicating the presence of species-specific antigens in the test sample within 15 minutes. In a preferred test procedure, a test sample is allowed to migrate out of the absorbent area or wick of the support and into the absorbent area that contains the conjugate pad. If a first antigen is present, labeled antibody-gold binds it, forming a first gold-antibody-antigen complex. As the reaction mixture continues to flow along the support, the first complex binds to another antibody immobilized in a visualization area producing a red colored band or line. Unbound conjugate binds to the reagents immobilized in a separate control area producing a red or pink colored band or line demonstrating proper performance of the test. This can be accomplished if the control area contains an immobilized reagent specific for the detectable conjugate reagent (e.g. an anti-immunoglobulin antibody) or by immobilizing analyte at the control area for which the detectable conjugate reagent is specific.

The present invention also provides methods and kits whereby more than one arthropod-borne agent can be detected essentially simultaneously using a single liquid permeable support. Also referred to as a panel assay, such multiple analyte detection system is accomplished by immobilizing an analyte-specific reagent for each arthropod-borne agent to be detected onto separate areas of the support and then contacting the sample with one or more detectable analyte-specific reagents, the mixture containing analyte-specific reagents, at least one specific for an analyte associated with each arthropod-borne agent to which the capture reagents are directed. The method can be adopted to detect two, three, four, five, six or more analytes on a single support, which can translate to detection of one, two, three, four, five, six or more different disease causing agents.

There are several factors that are important to consider when developing a panel assay for arthropod-borne pathogens, such as a Plasmodium circumsporozoite panel assay which has optimal charateristics. First, the analyte specific reagents need to be compatible as reagents for the assay. Analyte specific reagents such as antibodies need to be generated from similar, i.e. non-cross reactive, animal species. The use of monoclonal antibody provides a solution for a number of non-compatibility issues between reagents.

Also, the quantity of antibody associated with gold particles of each analyte-specific reagent and the ratio of the different gold-antibody complexes that are mixed together and applied to the conjugate pad are factors that effect the performance of the assay. For example, one may need to determine the most appropriate ratio of different colloidal gold conjugates to be used in construction of a multi-analyte detection strip (panel assay) to acquire the requisite sensitivity and specificity for each of the analytes in the panel. Depending on the type of assay and the reagents, one of the above factors may be more important than the other and evaluations need to be made to determine the optimum method.

In addition, the quantity of analyte specific reagent immobilized on the membrane is important for performance. The number of test lines and the particular sequence of the test lines created by immobilizing antibody perpendicular to the direction of flow of liquid on the test strip effects performance of the panel. This may be critical in a panel assay due to the chance that some of the reagent-analtye interactions are competitive. One should test for this and determine in prototype constructions and adjust the relative order of immobilized detection zones on the strip t to alleviate the crossreactivity or competitiveness of the reagents.

Furthermore, it is important for performance to determine that the extraction buffer is compatible with the other test reagents and the materials that comprises the test. The membrane pore size, its flow characteristics, the wick, conjugate and absorbent pad materials should be selected taking into consideration the variability of mosquito extracts and mosquito types intended to be screened.

By following the above teachings, one can prepare panel assays that effectively provide detection of multiple analytes and a level of sensitivity comparable to circumsporozoite ELISA in a single assay strip. Using such an approach, the present method of detecting arthropod-borne agents is capable of achieving a sensitivity and specificity of 90% or greater, more preferably a sensitivity and specificity of 95% or greater, and most preferably, a sensitivity and specificity of 98% or greater compared with circumsporozoite ELISA performed as described by the CDC.

The above method for multiple analyte can detect multiple analytes of a single disease-causing agent or multiple analytes, each from different disease-causing agents. In the latter case, detection of a genus may be combined with detection of species known to cause disease. For example, in malaria, P. falciparum and P. vivax or P. malariae or P. ovale are sometimes present together in endemic areas such as Cameron, Africa. The advantages of combining genus detection with species identification avoids additional work associated with parallel testing.

Preferably, the colored analyte-specific reagents that bind to said analytes are deposited on a portion of the support such as a conjugate pad, prior to contacting the support with the arthropod sample. These reagents may be deposited by methods known to those of ordinary skill in the art, including by imprinting, stamping or spraying as a fine mist onto the support or any other suitable means.

Information about the presence of a combination of organisms and their relative abundance can be useful in designing suppression and prevention plans. The methods of the present invention include the detection of malaria-causing microorganisms, preferably P. falciparum, P. vivax 210, and P. vivax 247. Thus simultaneously detection of a primary Plasmodium organism, i.e. a particular species or subspecies of Plasmodium, which is known to be an important disease-causing pathogen in the region from which the sample was taken, together with a less significant disease-causing pathogen, such as a secondary Plasmodium organism in accordance with the present invention should assist in designing suppression and prevention plans. As used herein, a primary pathogen is one that is endemic in the area while a secondary pathogen is one that is suspected of being present but not endemic (Vaughn et al., Am. J. Trop. Med. Hyg., 60(4):693-698 (1999).

The method also can include detection of P. vivax isolates other than Pv210 or Pv247 as these agents become known in the future. Such new species or types of Plasmodium can be included in the assay following the teachings disclosed herein.

The simultaneous detection of other useful combinations of arthropod-borne agents on a single support is both possible and useful. This includes, for example, two or more togaviruses or flaviviruses including encephalitis or dengue virus.

Another embodiment of the present invention are kits that include assay strips for using the methods described above. Such kits include detection of single arthropod-borne agents or multiple arthropod-borne agents.

The present invention also provides a novel clip-on construction for a container used in assays and kits for detecting disease-causing agents in arthropods. In this embodiment, the kit or assay comprises a container for the arthropod sample having an opening at one end covered with a filter, said container being adapted to clip onto a support containing a detection area. The construction allows the arthropod sample to contact the support while the filter prevents debris from the arthropods from migrating into the support. A preferred embodiment of the clip-on container relates to a lateral-flow format assay shown in FIG. 3. This format has a unique filter that can remove cellular debris or particulate matter and allow the immunochromatography process to take place in a cleaner background.

The arthropod-carried agents to be detected by the present invention include disease-causing pathogens, viruses, and vectors. Specific agents are discussed separately below.

A. Application of the Invention to Detection of Malaria

The causative agent of malaria is a protozoan of the genus Plasmodium. The four species of Plasmodium that are responsible for disease in humans are P. falciparum, P. vivax, P. ovale and P. malariae. The life cycle of the four species is generally similar and consists of two discrete phases: asexual and sexual. The asexual stages develop in humans, first in the liver and then in the circulating erythrocytes; the sexual stages develop in the mosquito.

In general, malarial infection is initiated by the injection of sporozoites into the bloodstream during a mosquito blood meal. Sporozoites rapidly disappear from the bloodstream as they invade the hepatic cells during passage through the liver. Within liver cells, the sporozoite rapidly differentiates into an intracellular form that undergoes asexual multiplication. One sporozoite can produce up to 20,000 parasites (i.e. merozoites) in this process. Clinical disease is initiated when merozoites are released from liver cells and invade reticulocytes and/or erythrocytes.

The four human malaria parasites can be differentiated by the properties of their asexual blood-stage infection and some aspects of parasite morphology. Asexual blood-stage malarial parasites cannot infect mosquitoes. The mosquito-infective forms are sexual forms of the malarial parasite, male and female gametocytes, that develop in infected erythrocytes.

The asexual blood-stage parasite is haploid; sexual differentiation does not involve nuclear division. Male and female gametocytes develop into large parasites that almost completely fill the infected erythrocyte. Gametocyte-infected erythrocytes often remain in the circulation for prolonged periods during which the levels of asexual parasites may wane.

The blood meal eaten by mosquitoes from a malaria infected individual includes uninfected erythrocytes, erythrocytes containing asexual parasites, and gametocyte-infected cells. However, only the gametocytes survive digestion in the mosquito gut. The host membrane surrounding these sexual stages is ruptured to release a large female gamete and slender, motile male gametes (gametogenesis). The male gametes fertilize the female gametes to produce a diploid zygote. The conversion of intracellular gametocytes to extracellular gametes and fertilization to form a zygote is largely completed within 30 minutes of blood ingestion. Zygotes remain within the contents of the blood meal for about 24 hr during which they transform into motile ookinetes. Mature ookinetes cross the mosquito midgut wall and continue development to form an oocyst. These grow and divide to produce many sporozoites which migrate to the mosquito salivary glands from where they enter the vertebrate host during mosquito feeding (Howard et al., “Malaria: Antigens and Host-Parasite Interactions” in Parasite Antigens, T. Pearson, ed., pp. 111-165, Marcel-Dekker Publishers, NY 1986).

The detailed symptomology and pathology of the human malarias have been reviewed (Miller, Transmissible Disease and Blood Transfusion, T. Greenwait and G. Jamieson, eds. Grune and Stratton, NY, 1975).

The methods or kits described herein include reagents specific for malarial analytes, preferably analytes associated with Plasmodium sporzoite, more preferably Plasmodium falciparum (Pf) circumsporozoite antigens, Plasmodium vivax (Pv) 210 and Pv247 circumsporozoite antigens. Preferred analyte-specific reagents for malaria include monoclonal antibodies that bind specifically with Pf, Pv210, and Pv247.

B. Application of the Invention to Detection of Dengue and Flaviviruses

Dengue and dengue associated hemorrhagic fever occur in epidemic form throughout the tropical areas of world. Dengue virus serotypes 1 through 4 have commonly been assayed using serological tests (hemaglutination-inhibition, immunofluorescence and complement fixation) with varying degrees of success. Presently, the only certain method of identification requires the use of standardized reference antiserum in a virus plaque-reduction neutralization assay. Since few field laboratories possess sufficient resources to perform this test with the slowly replicating dengue viruses, new methods are necessary. With the development of monoclonal antibodies against dengue antigens with demonstrated separate specificities for a flavivirus group-common, dengue-complex, sub-complex, and serotype-specific determinants opens the possibility of developing rapid assays to identify the virus strains (Henchal et al., Am. J. Trop. Med. Hyg., 32(1): 164-169, 1983).

Preferred Dengue virus analytes are those bound by antibodies produced by the cell lines from the American Type Culture Collection (“ATCC”) designated ATCC HB 114 (D3-2H2-9-21), ATCC HB 112 (D1-4G2-4-15), ATCC HB 46 (3H5-1), ATCC HB 47 (15F3-1), ATCC HB 48 (H110-6), and ATCC HB 49 (5D4-11).

C. Application of the Invention to Detection of Arboviruses

Arthropod-borne viruses, i.e. arboviruses, are viruses that are maintained in nature through biological transmission between susceptible vertebrate hosts by blood feeding arthropods (mosquitoes, psychodids, ceratopogonids, and ticks). Vertebrate infection occurs when the infected arthropod takes a blood meal. The term ‘arbovirus’ has no taxonomic significance.

Arboviruses that cause human encephalitis are members of three virus families: the Togaviridae, Flaviviridae, and Bunyaviridae. The Togaviridae family includes the Alphaviruses (arbovirus group A) such as Eastern and Western Equine Encephalitis viruses, and the Favivirus (arbovirus group B), including dengue virus, St. Louis Encephalitis and West Nile fever (see, e.g., Taxonomy of Viruses by Joseph Melnick, Chapter 62).

All arboviral encephalitides are zoonotic, being maintained in complex life cycles involving a nonhuman primary vertebrate host and a primary arthropod vector. These cycles usually remain undetected until humans encroach on a natural focus, or the virus escapes this focus via a secondary vector or vertebrate host as the result of some ecologic change. Humans and domestic animals can develop clinical illness but usually are “dead-end” hosts because they do not produce significant viremia, and do not contribute to the transmission cycle. Many arboviruses that cause encephalitis have a variety of different vertebrate hosts and some are transmitted by more than one vector. Maintenance of the viruses in nature may be facilitated by vertical transmission (e.g., the virus is transmitted from the female through the eggs to the offspring).

Arboviral encephalitides have a global distribution, but there are four main virus agents of encephalitis in the United States: Eastern Equine Encephalitis (EEE), Western Equine Encephalitis (WEE), St. Louis Encephalitis (SLE) and LaCrosse (LAC) Encephalitis, all of which are transmitted by mosquitoes. Another virus, Powassan, is a minor cause of encephalitis in the northern United States, and is transmitted by ticks. A new Powassan-like virus has recently been isolated from deer ticks. Its relatedness to Powassan virus and its ability to cause disease has not been well documented.

The majority of human arboviral infections are presumed to be asymptomatic or minor, resulting in a nonspecific flu-like syndrome. Onset may be insidious or sudden with fever, headache, myalgias, malaise and occasionally prostration. Infection may, however, lead to encephalitis, with a fatal outcome or permanent neurologic sequelae.

Experimental studies have shown that invasion of the central nervous system (CNS) generally follows initial virus replication in various peripheral sites and a period of viremia. Viral transfer from the blood to the CNS through the olfactory tract has been suggested. Because the arboviral encephalitides are viral diseases, antibiotics are not effective for treatment and no effective antiviral drugs have yet been discovered. Treatment is supportive, attempting to deal with problems such as swelling of the brain, loss of the automatic breathing activity of the brain and other treatable complications like bacterial pneumonia.

Eastern equine encephalitis (EEE), caused by a virus transmitted to humans and equines by the bite of an infected mosquito, is an alphavirus that was first identified in the 1930's and currently occurs in focal locations along the eastern seaboard, the Gulf Coast and some inland Midwestern locations of the United States.

The alphavirus Western equine encephalitis (WEE) was first isolated in California in 1930 from the brain of a horse with encephalitis, and remains an important cause of encephalitis in horses and humans in North America, mainly in western parts of the USA and Canada. In the western United States, the enzootic cycle of WEE involves passerine birds, in which the infection is inapparent, and culicine mosquitoes, principally Cx. tarsalis, a species that is associated with irrigated agriculture and stream drainages. The virus has also been isolated from a variety of mammal species. Other important mosquito vector species include Aedes melanimon in California, Ae. dorsalis in Utah and New Mexico and Ae. campestris in New Mexico. WEE virus was isolated from field collected larvae of Ae. dorsalis, providing evidence that vertical transmission may play an important role in the maintenance cycle of an alphavirus.

St. Louis encephalitis (SLE) is the leading cause of epidemic flaviviral encephalitis in the United States and the most common mosquito-transmitted human pathogen in the U.S. While periodic SLE epidemics have occurred only in the Midwest and southeast, SLE virus is distributed throughout the lower 48 states.

Since 1964, there have been 4,437 confirmed cases of SLE with an average of 193 cases per year (range 4-1,967). However, less than 1% of SLE viral infections are clinically apparent and the vast majority of infections remain undiagnosed. Illness ranges in severity from a simple febrile headache to meningoencephalitis, with an overall case-fatality ratio of 5-15%. During the summer season, SLE virus is maintained in a mosquito-bird-mosquito cycle, with periodic amplification by peridomestic birds and Culex mosquitoes. In Florida, the principal vector is Cx. nigripalpus, in the Midwest, Cx. Pipiens Pipiens and Cx. p. quinquefasciatus and in the western United States, Cx. tarsalis and members of the Cx. pipiens complex.

LaCrosse (LAC) encephalitis was discovered in LaCrosse, Wis. in 1963. Since then, the virus has been identified in several Midwestern and mid-Atlantic states. During an average year, about 75 cases of LAC encephalitis are reported to the Center for Disease Control (“CDC”). Most cases of LAC encephalitis occur in children under 16 years of age. LAC virus is a Bunyavirus and is a zoonotic pathogen cycled between the daytime-biting treehole mosquito, Aedes triseriatus, and vertebrate amplifier hosts (chipmunks, tree squirrels) in deciduous forest habitats. The virus is maintained over the winter by transovarial transmission in mosquito eggs. If the female mosquito is infected, she may lay eggs that carry the virus, and the adults coming from those eggs may be able to transmit the virus to chipmunks and to humans.

Powassan (POW) virus is a flavivirus and currently the only well documented tick-borne transmitted arbovirus occurring in the United States and Canada. Recently a Powassan-like virus was isolated from the deer tick, Ixodes scapularis. Its relationship to POW and its ability to cause human disease has not been fully elucidated. POW's range in the United States is primarily in the upper tier States. In addition to isolations from man, the virus has been recovered from ticks (Ixodes marxi, I. cookei and Dermacentor andersoni) and from the tissues of a skunk (Spiligale putorius). It is a rare cause of acute viral encephalitis. POW virus was first isolated from the brain of a 5-year-old child who died in Ontario in 1958. Patients who recover may have residual neurological problems.

Like EEE and WEE viruses, Venezuelan equine encephalitis (VEE) is an alphavirus and causes encephalitis in horses and humans and is an important veterinary and public health problem in Central and South America. Occasionally, large regional epizootics and epidemics can occur resulting in thousands of equine and human infections. Epizootic strains of VEE virus can infect and be transmitted by a large number of mosquito species. The natural reservoir host for the epizootic strains is not known. A large epizootic that began in South America in 1969 reached Texas in 1971. It was estimated that over 200,000 horses died in that outbreak, which was controlled by a massive equine vaccination program using an experimental live attenuated VEE vaccine. There were several thousand human infections. A more recent VEE epidemic occurred in the fall of 1995 in Venezuela and Colombia with an estimated 90,000 human infections. Infection of man with VEE virus is less severe than with EEE and WEE viruses, and fatalities are rare. Adults usually develop only an influenza-like illness, and overt encephalitis is usually confined to children. Effective VEE virus vaccines are available for equines.

Japanese encephalitis (JE) virus is a flavivirus, related to SLE, and is widespread throughout Asia. Worldwide, it is the most important cause of arboviral encephalitis with over 45,000 cases reported annually. In recent years, JE virus has expanded its geographic distribution with outbreaks in the Pacific. Epidemics occur in late summer in temperate regions, but the infection is enzootic and occurs throughout the year in many tropical areas of Asia. The virus is maintained in a cycle involving culicine mosquitoes and waterbirds. The virus is transmitted to man by Culex mosquitoes, primarily Cx. tritaeniorhynchus, which breed in rice fields. Pigs are the main amplifying hosts of JE virus in peridomestic environments.

Tick-borne encephalitis (TBE) is caused by two closely related flaviviruses which are distinct biologically. The eastern subtype causes Russian spring-summer encephalitis (RSSE) and is transmitted by Ixodes persulcatus, whereas the western subtype is transmitted by Ixodes ricinus and causes Central European encephalitis (CEE). The name CEE is somewhat misleading, since the condition can occur throughout much of Europe. Of the two subtypes, RSSE is the more severe infection, having a mortality of up to 25% in some outbreaks, whereas mortality in CEE seldom exceeds 5%.

West Nile encephalitis (WNV) is a flavivirus belonging taxonomically to the Japanese encephalitis serocomplex that includes the closely related St. Louis encephalitis (SLE) virus, Kunjin and Murray Valley encephalitis viruses, as well as others. WNV was first isolated in the West Nile Province of Uganda in 1937 (2). The first recorded epidemics occurred in Israel during 1951-1954 and in 1957. Epidemics have been reported in Europe in the Rhone delta of France in 1962 and in Romania in 1996 (3-5). The largest recorded epidemic occurred in South Africa in 1974 (6).

### WEST NILE ENCEPHALITIS REFERENCES

1. CDC. Case definitions for infectious conditions under public health surveillance. MMWR 1997;46(RR-10): 12-3.

2. Smithburn K C, Hughes T P, Burke A W, Paul J H. A neurotropic virus isolated from the blood of a native of Uganda. Am J Trop Med Hyg 1940;20:471.

3. Klingberg M A, Jasinka-Klingberg W, Goldblum N. Certain aspects of the epidemiology and distribution of immunity of West Nile virus in Israel. In: Proceeding of the 6th International Congress of Tropical Medicine, 1959;5:132.

4. Panther R, Hannoun C, Beytout D, Mouchet J. Epidemiology of West Nile virus. In: Human Illness: focus on Camargue [French]. Vol 3. Ann Inst Pasteur 1968;115:435.

5. Tsai T F, Popovici F, Cernescu C, Campbell GL, Nedelcu N I. West Nile encephalitis epidemic in southeastern Romania. Lancet 1998;352:767-71.

6. McIntosh B M, Jupp P G, Dos Santos I, Meenehan G M. Epidemics of West Nile and Sindbis viruses in South Africa with Culex (Culex) univittatus Theobold as vector. S Afr J Sci 1976;72:295.

Murray Valley encephalitis (MVE) is endemic in New Guinea and in parts of Australia; and is related to SLE, WN and JE viruses. Inapparent infections are common, and the small number of fatalities have mostly been in children.

D. Application of the Invention to Detection of Ross River Virus

Ross River virus infection is a viral infection that occurs in all States in Australia. It is an Arbovirus of the Alphavirus genus. It can cause a wide range of infections, the most serious is arthritis, usually in the writs, knees and ankles. The virus is spread to humans by mosquitoes. Ross River virus and specific antibodies thereto are available from the American Type Culture Collection (e.g., antibody to strain T-48: ATCC VR-1246 and Ross River virus strain T-48: ATCC VR-373).

The invention is further illustrated by, though in no way limited to, the following examples.

### EXAMPLES

### Example 1

### Selection of Antibodies

Antibodies to Plasmodium, togaviruses, flavivirues and Ross River virus described herein and other viruses are available from various public sources including, for example, the American Type Culture Collection (Rockville, Md.) (“ATCC”) or Center for Disease Control (Fort Collins, Calif.) (“CDC”) as indicated in Table 1.

TABLE 1
Antibodies for Detecting Arthropod-Borne Diseases

| | | |
|---|---|---|
| Antibody/ | | |
| Cell Line | Specific to | Reference/Source |

Flavivirus

| | | |
|---|---|---|
| D1-4G2-4-15 | Flavivirus | ATCC |

Dengue

| | | |
|---|---|---|
| D3-2H2-9-15 | Dengue 1-4 | ATCC |
| D2-15F3-1-15 | Dengue 1 | ATCC/Am. J. Trop. Med. Hyg., |
| | | Henchal et al, 32(1) 164-169 |
| 1F1-3 | Dengue 1 | WRAIR/AFIRMS - Thailand |
| D1-3H5-1-21 | Dengue 2 | ATCC/Am. J. Trop. Med. Hyg., |
| | | Henchal et al, 32(1) 164-169 |
| 5D4-11 | Dengue 3 | ATCC |
| 10C10-5 | Dengue 3 | WRAIR/AFIRMS - Thailand |
| D3-1H10-6-7 | Dengue 4 | ATCC/Am. J. Trop. Med. Hyg., |
| | | Henchal et al, 32(1) 164-169 |

Malaria

| | | |
|---|---|---|
| Pf2A10 | P. falciparum | Wirtz et al, Bull WHO 65:39-45 |
| | | (1987) |
| Pf1B2.2 | P. falciparum | Wirtz et al, Bull WHO 65:39-45 |
| | | (1987) |
| NSV#3 | Pv210 | Wirtz et al, Am. J. Trop. Med. |
| | | Hyg., 34: 1048-1054 (1986) |
| 2E10 | Pv247 | Wirtz et al, J. Med. Entomol., 29: |
| | | 854-57 (1992) |

Encephalitis

| | | |
|---|---|---|
| 1A4B-6 | Broad alphavirus | CDC |
| 1B5C-3 | Eastern Equine | CDC |
| | Encephalitis | |
| | (“EEE”) | |
| 1B1C-4 | Eastern Equine | CDC |
| | Encephalitis | |
| | (N. & S. American | |
| | specific) | |
| 2A3D-5 | Western Equine | CDC |
| | Encephalitis | |
| | (“WEE”) | |
| 2B1C-6 | Western Equine | CDC |
| | Encephalitis | |
| 4A4C-4 | St. Louis | CDC |
| | Encephalitis | |
| | (“SLE”) | |
| 6B6C-1 | Flavivirus | CDC |
| | cross-reactive | |

Ross River Virus

| | | |
|---|---|---|
| VR-1246 | Ross River Virus | ATCC |
| VR-373 | Ross River Virus | ATCC |

The following combination of antibodies and positive controls were used:

A. Malaria Test:

Plasmodium falciparum

Mab Pf2A10 (474) Refer to #1 below

Mab Pf1B2.2 (93-3-5) Refer to #1 below

Positive analyte control Pf-R32tet32 (104189) Refer to #1 below

Plasmodium vivax

Mab Pv-210 (NSV#3) Refer to #2 below

Positive analyte control Pv-210 (105232) Refer to #2 below

Mab Pv-247 (1G12.1) Refer to #3 below

Positive analyte control Pv-247 Refer to #3 below (synthetic peptide)

### MALARIAL REFERENCES

1. Wirtz et al. (1987) Comparative testing of Plasmodium falciparum sporozoite monoclonal antibodies for ELISA development. Bull. WHO 65:39-45.

2. Wirtz et al. (1986) Identification of Plasmodium vivax sporozoites in mosquitoes using an enzyme-linked immunosorbent assay. Am. J. Trop. Med. Hyg.34: 1048-1054.

3. Wirtz et al. (1992) Development and evaluation of an ELISA for Plasmodium vivax-VK247 sporozoites. J. Med. Entomol. 29: 29:854-57.

B. Encephalitis Tests:

Possible Encephalitis Antibody Pair Combinations:

SLE: 6B6C-1 (capture) and 4A4C-6 (gold label)

WEE: 2A3D-5 (capture) and 2B1C-6 (gold label)

EEE: 1B5C-3 (capture) and 1A4B-6 (gold label)

### Example 2

### Preparation of Detectably Labeled Antibody Reagents

This example describes a method for conjugating detectable analyte-specific reagents. Specifically disclosed is a method of conjugating an antibody to colloidal gold and to latex. Colloidal gold labeled antibodies were prepared essentially as described by Hermanson, Bioconjugate Techniques, Academic Press, 1996, volume 14. The method is briefly summarized below.

A. Preparation of Colloidal Gold

Mono-disperse colloidal gold suspensions were prepared using the reductive process on chloroauric acid (HAuC14) to create 15 to 35 run particles. Briefly, a 2% gold chloride stock solution (Sigma Chem. Co. or ICN pharmaceuticals) was made by adding 20 g of gold chloride powder (stock # GC-S) to 1000 mL distilled water and mixing. The stock was diluted to 0.01% with distilled water and heated with stirring to 90-100° C. At boil, add approximately 5 mL (for 200 mL) of the 1% sodium citrate solution to achieve the size of the desired size gold particles. After 10 minutes, the solution is quickly cooled to ˜60° C. using a condenser and the absorbance of the solution read from 574-474 nm on a spectrophotometer, using water as the background. The peak OD of the solution should be at 520 nm, a value which indicates 20-25 nm diameter particles. If the value is much different, the amount of sodium citrate is adjusted accordingly.

A. Preparation of Antibody-Colloidal Gold Complexes:

Freshly prepared 520 nm gold colloid solution, described above was used for conjugation. Optimal coupling was determined by the following criteria described in Hermanson, Bioconjugate Techinques, supra:

1. selection of optimal coupling pH which is dependent on the antibody pI (isoelectric point);

2. selection of buffer type and concentration to be used;

3. determination of minimum amount of antibody required to stabilize the colloidal gold being used;

4. time for reaction;

5. selecting method for further stabilizing with extraneous proteins/polymers;

6. selecting method for separating (centrifugation—time and speed); and

7. fixing composition of resuspension buffer, pH, concentration and stability verification.

In the examples described herein, antibody was typically adsorbed to colloidal gold using the procedure described in Beesley, Colloidal Gold: A New Perspective for Cytochemical Marking, Oxford University Press, 1989 and Hermanson, supra, pages 600-601. Conjugate was diluted in a stabilizing buffer, essentially as described by Beesley, supra, page 10, further including polyvinylpyrrolidone, sucrose and an appropriate non-ionic detergent.

B. Preparation of Latex Labeled Antibodies:

A. Particle type:

Carboxy modified died PS latex, 10% suspension IgG: 1 mg/ml solution in phosphate buffer.

B. Method of coupling:

One step EDAC mediated covalent coupling was used essentially as described in Hermanson, Bioconjugate Techniques, Academic Press, 1996.

### Example 3

### Preparation of Samples Containing Arthropod-Borne Agents

Arthropod-borne agents containing analytes of interest were obtained from, various sources summarized in the Table 2 below.

TABLE 2
Analytes for Arthropod-Borne Pathogens
Dengue

| | | Evaluated | |
|---|---|---|---|
| Antigen | Form | for | Reference/Source |
| Dengue 1 Westpack | Inactivated | Dengue 1 | WRAIR/AFRIMS, |
| 74 Vero P3 P5 | | | Thailand |
| culture 2/26/00 | | | |
| Dengue 2 S16803 | Inactivated | Dengue 2 | WRAIR/AFRIMS, |
| Vero P3 P5 culture | | | Thailand |
| 2/26/00 | | | |
| Dengue 3 CH53489 | Inactivated | Dengue 3 | WRAIR/AFRIMS, |
| Vero P3 P5 culture | | | Thailand |
| 2/26/00 | | | |
| Dengue 4 TVP360 | Inactivated | Dengue 4 | WRAIR/AFRIMS, |
| Vero P3 P5 culture | | | Thailand |
| 2/26/00 | | | |
| Dengue 2 antigen | Inactivated | Dengue 2 | Microbix, Toronto, |
| | | | Canada |
| Dengue 2 envelope | Recombinant | Dengue 2 | WRAIR/AFIRMS, |
| protein | | | Thailand |
| Dengue 2 virus | Inactivated | Dengue 2 | WRAIR/AFRIMS, |
| | | | Thailand |
| Dengue tetravalent | Inactivated | Dengue | WRAIR/AFRIMS, |
| complex (1996) | | 1, 2, 3, 4 | Thailand |
| Recombinant Den-3 | Lyophilized; | Dengue 3 | Hawaii Biotech, |
| 80% E | recombinant | | Aiea, HI |
| Recombinant Den-4 | Lyophilized; | Dengue 4 | Hawaii Biotech, |
| 80% E | recombinant | | Aiea, HI |
| Dengue-2 | Inactivated | Dengue 2 | Immunology |
| | | | Consultants |
| | | | Laboratory, Sherwood, |
| | | | OR |
| Aedes aegypti | Negative | N/A (known | WRAIR/AFRIMS, |
| mosquito | | dengue | Thailand |
| | | vector) | |
| Anopheles stephensi | Negative | N/A (known | WRAIR/AFRIMS, |
| | | malaria | Thailand |
| | | vector) | |

Malaria

| | | Evaluated | |
|---|---|---|---|
| Antigen | Form | for: | Reference/Source |
| Pf-R32tet32 | Recombinant | Pf | WRAIR, Wirtz et al., |
| | | | Bull. WHO, 65 (1), |
| | | | p 39-45 (1987) |
| Pv210 | Recombinant | Pv210 | Wirtz, et al, 1986, |
| | | | Am J. Trop. |
| | | | Med. Hyg., 34, |
| | | | 1048-1054 (1986) |
| | | | Rosenberg et al, |
| | | | Science, 245, |
| | | | 973-976 (1989) |
| Pv247 | Peptide | Pv247 | Wirtz, et al, 1992 |
| | conjugated | | |
| | to boiled | | |
| | casein | | |
| Anopheles stefensi | Positive | Pf | WRAIR |
| Anopheles stefensi | Positive | Pv210 | WRAIR |
| Anopheles gambiae | Negative | N/A (Known | WRAIR |
| | | malaria | |
| | | carrier) | |
| Anopheles stefensi | Negative | N/A (Known | WRAIR |
| | | malaria | |
| | | carrier) | |

Encephalitis

| Antigen | Form | Specific to | Reference/Source |
|---|---|---|---|
| NJ/60 Noninf. AGN | Inactivated | Eastern | CDC/Dr. Nasci |
| SASMB | | equine | |
| | | encephalitis | |
| Fleming Noninf. | Inactivated | Western | CDC/Dr. Nasci |
| AGN SASMB | | equine | |
| | | encephalitis | |
| TBH-28 Noninf. | Inactivated | St. Louis | CDC/Dr. Nasci |
| AGN SASMB | | Encephalitis | |

Samples of mosquitoes were obtained by grinding the arthropods in boiled casein blocking solution, referred to herein as “mosquito grinding solution” A one liter batch of grinding solution includes 900 ml of phosphate buffered saline pH, 7.4 (containing 0.2 g/L KCl) (“PBS”), 100 ml of 0.1N NaOH, 5 g casein (Sigma, Chem Co., C-7078), 50 ml of an appropriate detergent such as NP-40, Tween-20 or Triton X-100 (10% stock) and 2.5 ml sodium azide (20% stock).

### Example 4

### Preparation of Dipsticks

Dipsticks used in the examples that follow were prepared using essentially the following approach.

1. Conjugate Pad

a. Dilute Ab*gold conjugate with stabilizing buffer to the appropriate antibody concentration (determined empirically).

b. Evenly apply conjugate to glass fiber material (Whatman). From 1 to 2 ml conjugate is absorbed by 1×30 cm conjugate pads.

c. Allow to air dry at room temperature and then dry further at 37-45° C. for 10-20 min using a fan.

2. Membrane

a. Use 8 μm pore size, 25 mm×50 m role of nitrocellulose as the membrane (Schleicher and Schull).

b. Print the different antibody solutions on the membrane using IVEK Digispense 2000™ printing machine (Springfield, Vt.) with four pump-heads, generally in accordance with the manufacturers instructions.

i. Print control line (C) and other test lines (e.g., T1, T2, T3). Program the IVEK machine to print various lines using an appropriate volume so as to effect control and test line width.

ii. Use the proper concentration of each of the antibodies, which are diluted in 0.1M phosphate pH: 7 buffer (determined empirically). For example, the control line containing goat anti-mouse antibody is printed at 1.25 mg/ml. iii. The volume of printed antibody solutions is approximately 1-2 μl/cm.

c. Dry membrane at room temperature.

3. Prepare Card Format (Lamination)

a. Take adhesive backing (G&L Inc., San Jose, Calif.): 26 cm×8 cm for dipstick assay and remove cover.

b. Place (stick) printed membrane in the right position on adhesive card.

c. Place dried conjugate pad onto the bottom part of membrane.

d. Place a 26 cm×3.2 cm piece of glass fiber wick pad (Whatman) on the top of the conjugate pad.

e. Place 26 cm long absorbent pad (Whatman, 25 mm×50 m roll) onto the top part of membrane.

f. Place stickers on conjugate pad and absorbent pad.

4. Cut Strips from Card

a. Use Kinematic Automation PS-360 (Programmable Shear) cutting machine to cut strips.

b. Cut 4 mm wide strips and store dry at room temperature.

### Example 5

### Single Analyte Assay for Plasmodium Species Detection

This example describes various single analyte assays for detection of Plasmodium sporozoite associated analytes in an arthropod sample using a dipstick as the fluid permeable support. Different antibodies specific for Plasmodium falciparum sporozoites were used in the single analyte format as described below.

1. Pf2A10*Gold Assay (P. falciparum)

In this assay, the Pf2A10 monoclonal antibody to P. falciparum was used as both the analyte-specific capture reagent and the detectable analyte-specific reagent, which in this case was colloidal gold labeled (indicated as “*gold”). Dipsticks were prepared as described in Example 4, and the Pf2A10 antibody was printed on the membrane at a concentration of 0.65 mg/ml.

In the assay, 0.25 ml of various concentrations of P. falciparum antigen in PBS including 10 ng/ml, 1 ng/ml and 0.1 ng/ml antigen and further including 0.1% Tween-20 or 0.5% NP-40 to generate analyte control solutions. Also, a control solution was made with 0.25 ml PBS with either detergent. Each solution was placed into a 1.5 ml Ependorf centrifuge tube and the dipstick was inserted with the wick end into the tube so as to make contact with the solutions. The test was maintained at room temperature and the results were read visually between 10 and 30 minutes after start.

The control line (C) was positive in all dipsticks and the buffer-detergent tested dipsticks were negative at the test line (T). The test line (T) for the analyte control solutions for both detergents showed color, indicating detection of P. falciparum at 1 ng/ml of antigen but not at 0.1 ng/ml antigen. Thus, the sensitivity limit of this Plasmodium falciparum sporozoite assay is between 1 and 0.1 ng/ml.

The specificity of the Pf2A10*gold assay was evaluated by performing as above, but substituting P. falciparum antigen with P. vivax 210 or 247 antigen, each at 10 ug/ml. In this test the control line was positive in both dipsticks while the test line with Pv210 and Pv247 antigens at 10 μg/ml did not give any signal.

2. Pf1B2.2*Gold Assay (P. falciparum)

In this assay, dipsticks were prepared using the Pf1B2.2 monoclonal antibody (93-3-5) gold labeled in the conjugate pad. The Pf2A10 antibody (474) was again used as the capture antibody. The dipsticks were inserted into the same test solutions as above.

All tests developed a control line (C) and the buffer-detergent solution tested dipstick was negative at the test line (T). The test line (T) results for the analyte control solutions showed detection of P. falciparum antigen at concentrations as low as 1 ng/ml, although the signal is slightly less intense than at 10 ng/ml. The test line was negative with an analyte control solution containing P. vivax antigen 210 or 247, each at 10 ug/ml. Thus, the sensitivity limit of this Plasmodium falciparum circumsporozoite assay is between 1 and 0.1 ng/ml.

3. Pv210*Gold Assay (P. vivax 210)

a. Gold Labeled Antibody:

In these tests, dipsticks were prepared using the Pv210 specific monoclonal antibody NSV3, colloidal gold labeled, and applied to the conjugate pad. The Pv210 capture antibody (NSV3) was printed on the membrane at a concentration of 0.3 mg/ml.

Analyte control solutions of 10 ng/ml, 1 ng/ml and 0.1 ng/ml antigen were prepared with P. vivax 210 antigen in PBS with Tween-20 as described in the Pf2A10* Gold Assay above. In addition, a buffer-detergent solution was also used as a control.

All tests developed a control line (C) and the buffer-detergent tested dipstick was negative at the test line (T). The test line (T) results for the analyte positive control solutions showed detection of P. vivax 210 at antigen at 10 and 1 ng/ml with reduced but visible detection at 0.1 ng/ml. The test line was negative with an analtye solution containing P. falciparum antigen at 10 ug/ml. Thus, the sensitivity limit of this Plasmodium vivax sporozoite assay is about 0.1 ng/ml.

b. Latex-Labeled Antibody:

In this example, dipsticks were prepared using blue latex conjugated NSV3 (anti Pv210 antibodies) (Pv210*latex) added to the conjugate pad. The Pv210 capture antibody (NSV3) was printed on the membrane at a concentration of 0.3 mg/ml.

Analyte control solutions included 80 ng/ml, 10 ng/ml and 4 ng/ml Pv210 antigen or buffer-detergent without antigen were prepared as described above. The assay was otherwise performed essentially as described above for the Pf2A10* Gold Assay.

The control line (C) was positive in all dipsticks and the buffer-detergent tested dipstick was negative at the test line (T). The test line for analyte control solutions (T) showed color indicating detection of P. vivax at lower than 1 ng/ml of antigen. The latex results are summarized in the table below.

TABLE 2
Latex conjugate results for Detection of Plasmodium vivax (210)

| Test | Concentration of Test | Volume of Test | Results at 20 |
|---|---|---|---|
| Dipstick | Antigen (ng/ml) | Antigen (μl) | minutes |
| 1 | 80 | 200 | 3+ |
| 2 | 10 | 200 | 3+ |
| 3 | 4 | 200 | 2+ |
| 4 | 0 | 200 | No signal |

Using gold conjugate containing dipsticks (same antibody) there was a 3 + result at 4 ng/ml antigen Pv210 and 2+result for 1 ng/ml Pv210 antigen. Thus, the sensitivity for Pv210 antigen using latex conjugate was comparable to the sensitivity using gold conjugate.

4. Pv247*Gold Assay (P. vivax 247)

In these tests, dipsticks were prepared with Pv247 specific monoclonal antibody 2E10 colloidal gold label applied to the conjugate pad. The capture antibody also was 2E10 and was printed on the membrane at a concentration of 0.75 mg/ml.

The assay was evaluated using control analyte solutions containing P. vivax 247 antigen at 25, 10 and 1 ng/ml in PBS with either 0.5% NP-40 or 0.1% Tween-20. Also tested was a control solution with buffer and either detergent. The assay was otherwise performed essentially as described for the Pf2A10*Gold Assay above.

All tests developed a control line (C) and the buffer-detergent tested dipsticks were negative at the test line (T). The test line (T) for both NP-40 and Tween-20 analyte control solutions showed clear detection of P. vivax 247 antigen at 25 and 10 ng/ml, while the signal was visible but very weak at 1 ng/ml (similar to the Pv210*gold assay). The test line was negative with an analyte solution containing P. falciparum antigen at 10 ug/ml. Thus, the sensitivity limit of this Plasmodium vivax sporozoite assay is between 10 and 1 ng/ml.

### Example 6

### Multiple Analyte Assay for Plasmodium Species Detection

This example describes various multiple analyte assays for detection of Plasmodium species in an arthropod sample using a single dipstick as the fluid permeable support.

A. Combined Pv210 and Pf Dipstick Assay:

Panel assays were developed whereby P. vivax 210 and P. falciparum (Pf) were detected on the same dipstick. Three lines were printed on each dipstick including a control line and a test line for each capture antibody. The gold conjugates against each antigen were mixed in the conjugate pad.

| Printed lines: control | Gold conjugates - mixed: |
|---|---|
| Mab to Pv210 | Mab to Pv210*gold |
| Mab to Pf2A10 | Mab to Pf2A10*gold |

Combination antigen detection dipsticks were prepared using monoclonal antibody Pf2A10 specific for P. falciparum and monoclonal antibody NSV3, specific for Pv210. Both antibodies were labeled with colloidal gold. The same antibodies were used for the capture with the Pv210 antibody added proximal to the control line and antibody Pf2A10 added distal to the control line.

1. Assay for Pv 210 Antigen:

In this assay, the combination dipsticks were tested using analyte control solutions containing Pv210 antigen at 10 ng/ml, 4 ng/ml, 1 ng/ml, 0.25 ng/ml and 0 ng/ml (buffer only) in PBS with 0.5% NP-40 or 0.1% Tween-20. The assay was otherwise performed essentially as described for the Pf2A10*Gold Assay above.

All tests developed a control line (C) and the buffer-detergent tested dipsticks were negative at the test line (T). For both NP-40 and Tween-20 containing solutions, the test line PV (containing Pv210 specific capture antibody) results showed clear detection of P. vivax 210 antigen at all concentrations including down to 0.25 ng/ml. In contrast, the detection zone (Pf) for P. falciparum antigen was negative at all Pv210 antigen concentrations used with both detergents. Thus, the sensitivity limit of this combined Plasmodium falciparum-Plasmodium vivax 210 sporozoite assay is as low as 0.25 ng/ml Pv210 and there is no reaction of the Pv 210 antibody with P. falciparum. Thus, no non-specific Pf signal developed in these assays.

2. Assay for Pf Antigen:

In this assay, the combination dipsticks were tested for detection of P. falciparum (Pf) antigen at 500 pg/ml, 100 pg/mil, 50 pg/ml, 5 pg/ml and 0 ng/ml (buffer only) in PBS containing either NP-40 or Tween-20 as above. The assay was otherwise performed essentially as described for the Pf2A10*Gold Assay above.

All tests developed a control line (C) and the buffer-detergent tested dipsticks were negative at the test line (T). For both types of detergent containing solutions, the test line (Pf) containing immobilized antibodies specific for Pf antigen showed clear detection of P. falicpirum antigen down to 100 pg/ml. In contrast, the test line (Pv) for P. vivax 210 antigen was negative at all Pf antigen concentrations used for both detergents. Thus, the sensitivity limit of this combined Plasmodium falciparum-Plasmodium vivax 210 sporozoite assay is as low as 100 pg/ml for Pf antigen and there is no reaction of the Pf specific antibody with P. vivax 210. Thus, when Pf antigen is tested on the Pf+Pv combo test dipstick, only the specific Pf signal is seen.

3. Assay for a Mixture of Pv 210 and Pf Antigen:

In this assay, the combination dipsticks were tested against a single control analyte solution containing either P. falciparum (Pf) antigen at 250 pg/mil and Pv210 antigen at 5 ng/ml or P. falciparum (Pf) antigen at 50 pg/ml and Pv210 antigen at 2 ng/ml all in PBS with either NP-40 or Tween-20 as above. The assay was otherwise performed essentially as described for the Pf2A10*Gold Assay above.

All tests developed a control line (C) and the buffer-detergent tested dipsticks were negative at the test line (T). For both detergents, the antigen mixtures gave detection results comparable to those achieved in individual assays with both antigens detected at each test line for the two mixtures used. The test line (Pf) containing immobilized antibodies specific for Pf antigen showed clear detection of P. falciparum antigen down to 100 pg/ml. In contrast, the test line (Pv) for P. vivax 210 antigen was negative at all Pf antigen concentrations used. Thus, the sensitivity is comparable (same) for both analytes (for both detergents) in the combined assay as is in the individual assays using the combination analyte test dipsticks.

B. Combined Pv210, Pv247 and Pf Dipstick Assay:

Panel assays were developed whereby P. vivax 210, P. vivax 247 and P. falciparum (Pf) can be detected on the same dipstick. Four lines were printed on each dipstick including a control line and a test line for each capture antibody. The gold conjugates against each antigen were mixed in the conjugate pad.

| Printed lines: control | Gold conjugates - mixed: |
|---|---|
| Mab Pf2A10 | Mab Pf2A10*gold |
| Mab Pv210 | Mab Pv210*gold |
| Mab Pv247 | Mab Pv247*gold |

Combination antigen detection dipsticks were prepared using monoclonal antibody Pf2A10 specific for P. falciparum, monoclonal antibody NSV3 specific for P. vivax 210 and monoclonal antibody 2E10 specific for P. vivax 247. The antibodies were labeled with colloidal gold for detection. The same antibodies were used for the capture with the Pf2A10 antibody added most proximal to the control line, the Pv210 antibody intermediate in position and the antibody Pv247 added most distal to the control line.

This assay was performed as the dual analyte combination dipsticks discussed above except that in this assay, the combination dipsticks were tested for detection against a mixture of all three antigens (Pf, Pv210 and Pv247), each at 12.5 ng/ml, 4.2 ng/ml, 0.8 ng/ml, 0.4 ng/ml, 0.2 ng/ml, 0.08 ng/ml and 0 ng/ml (buffer only) in PBS with 0.5% NP-40. A control solution with PBS and NP-40 also was used.

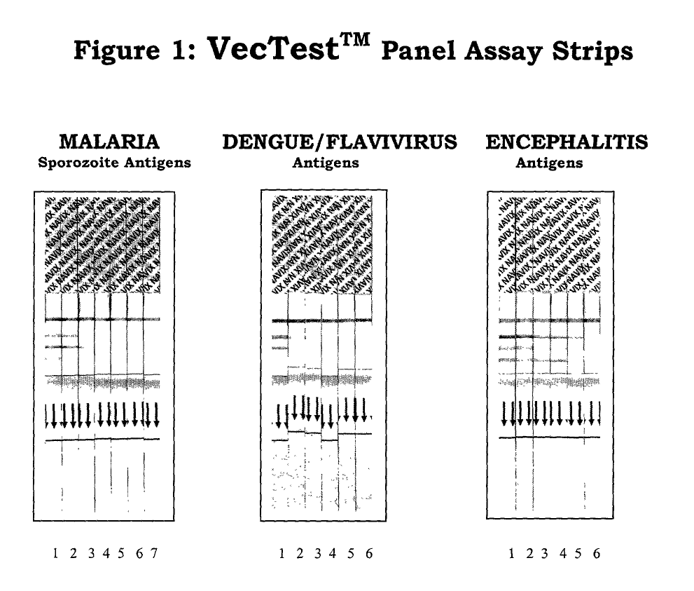

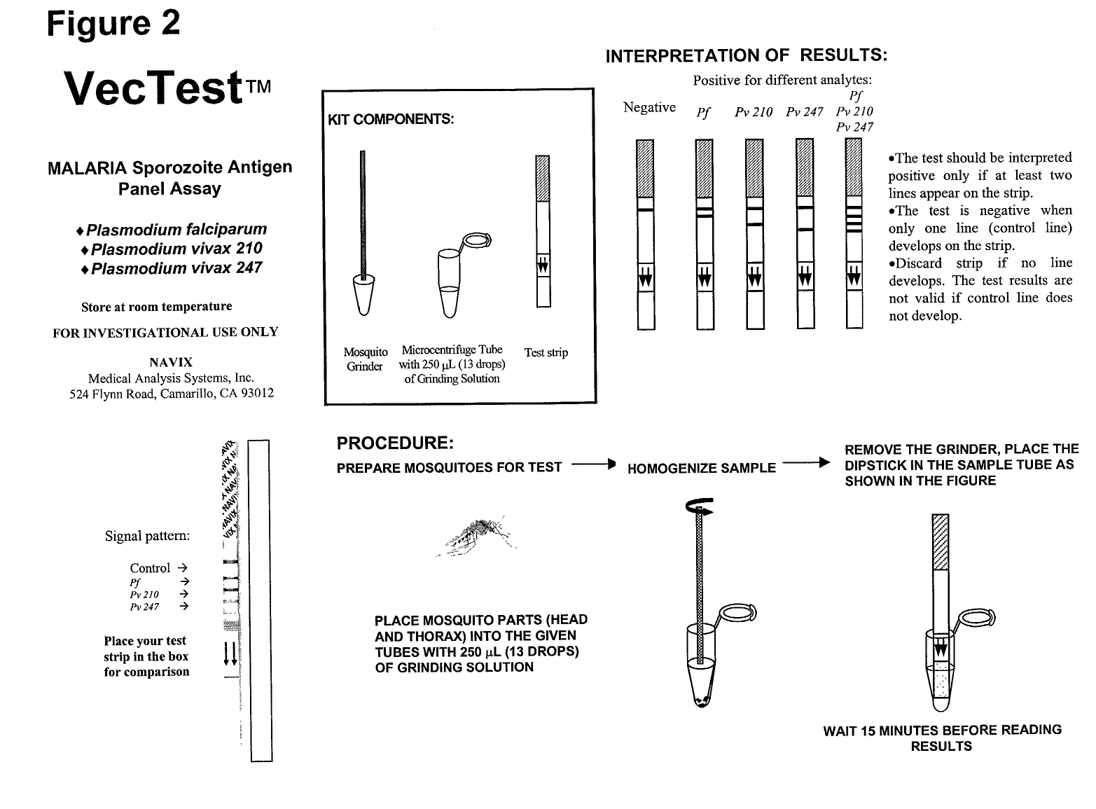

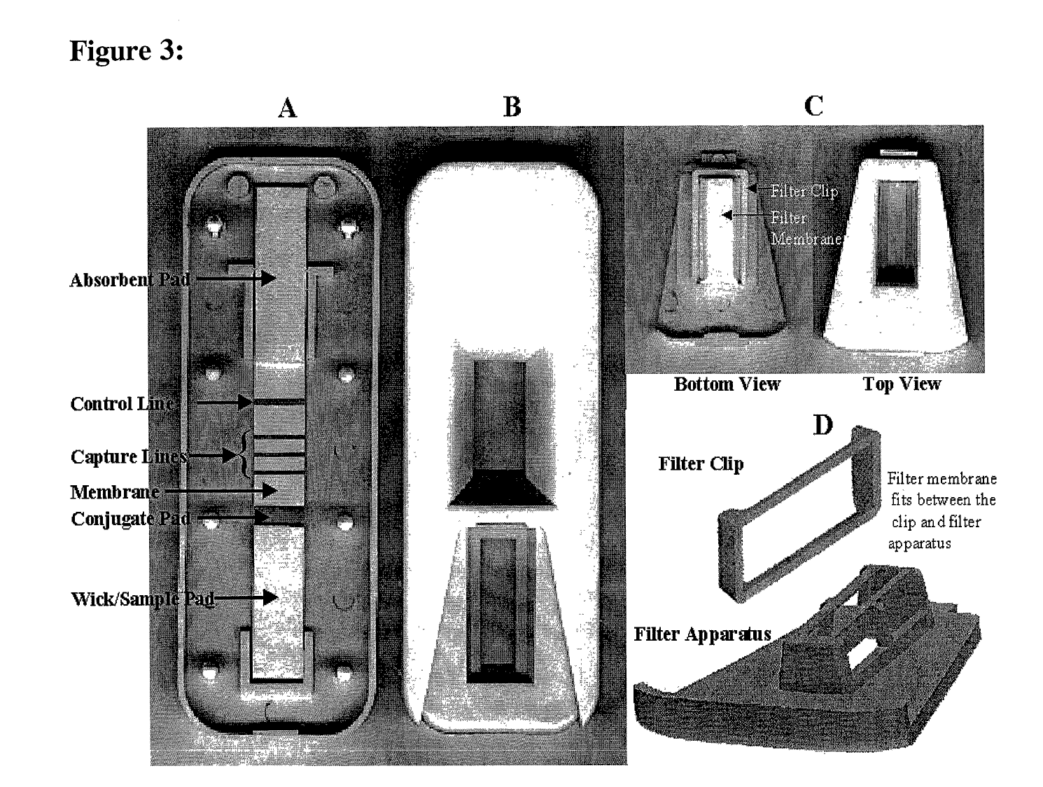

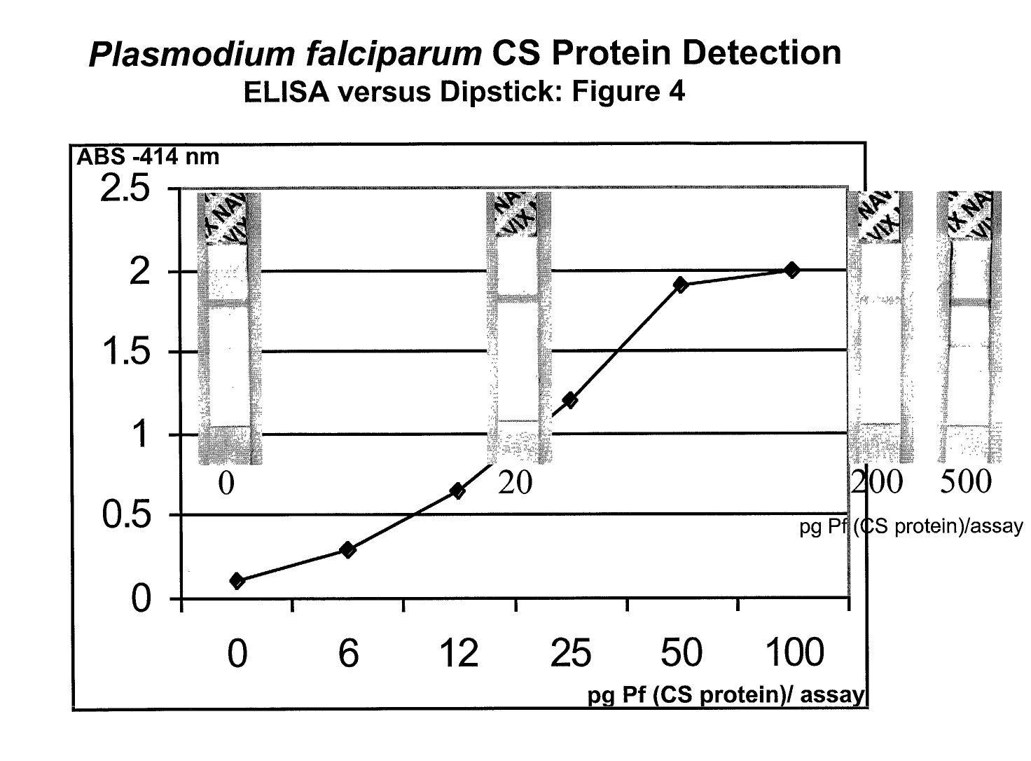

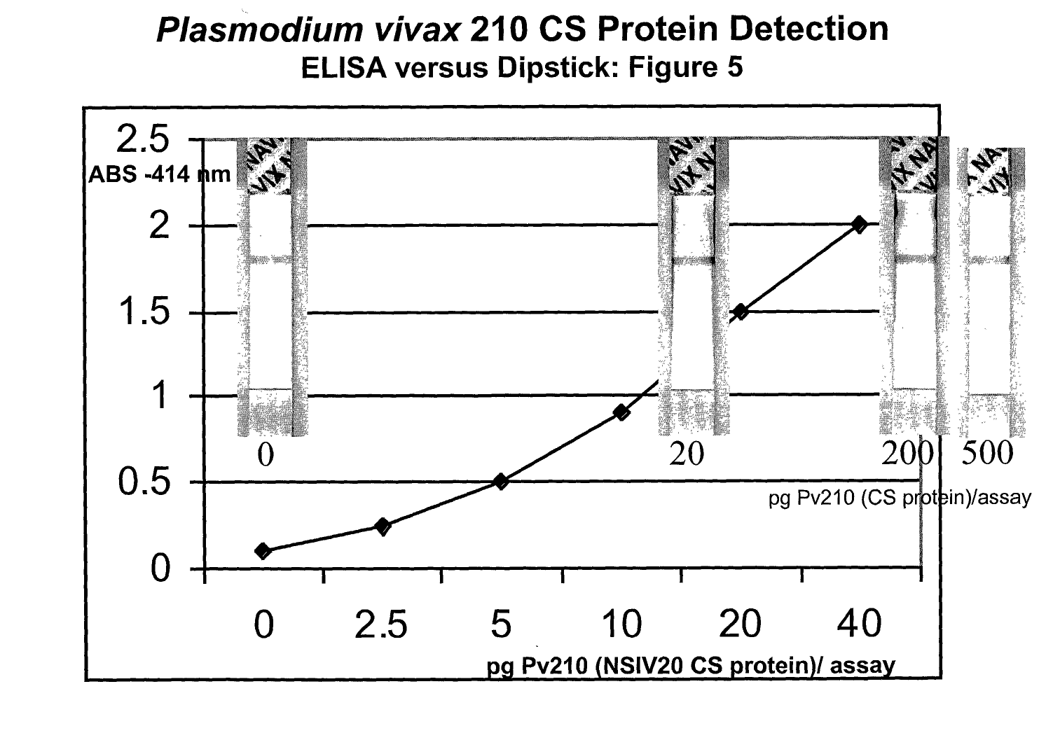

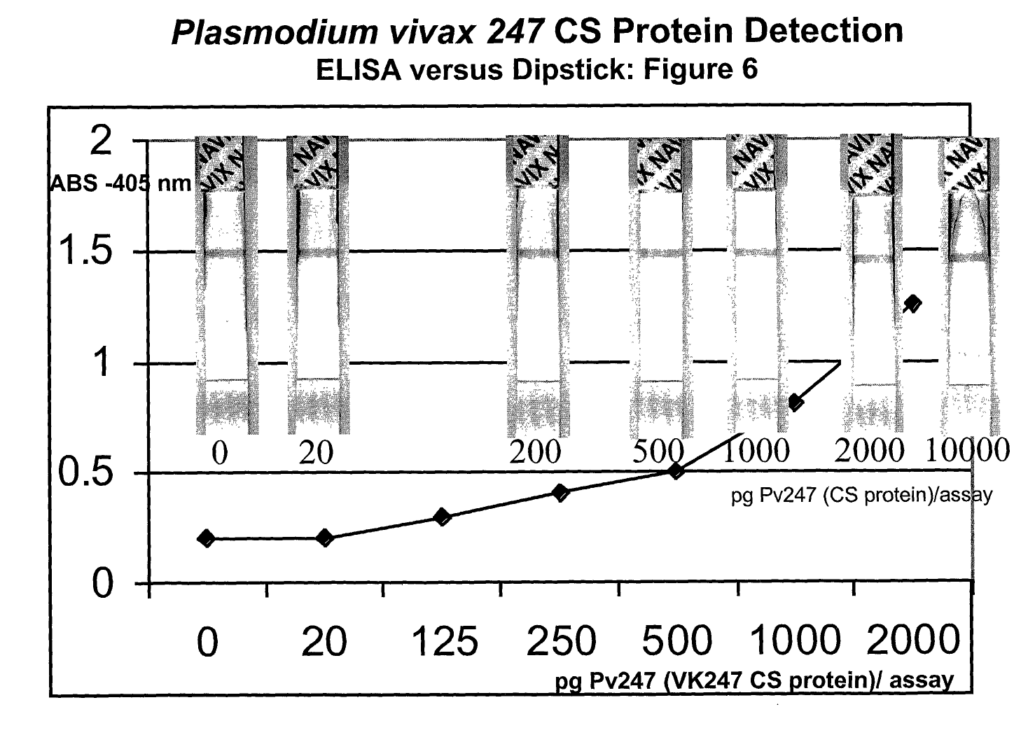

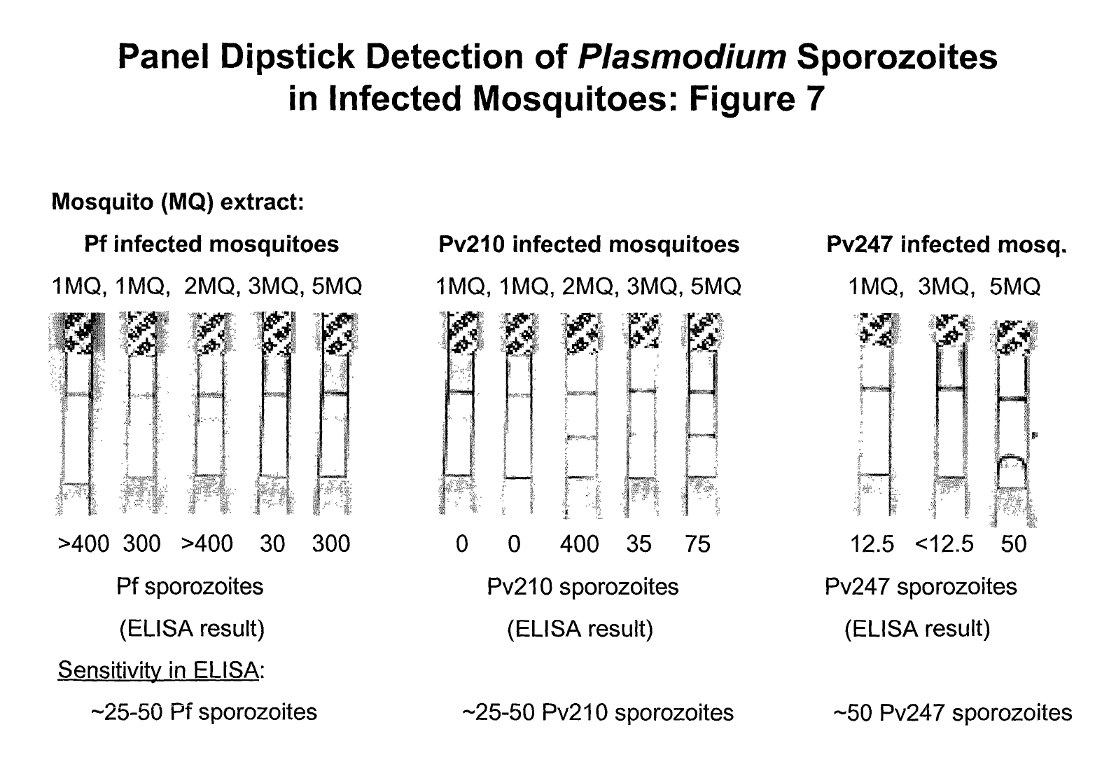

The results are shown in FIG. 1, panel A. All tests developed a control line (C) and the buffer-detergent tested dipstick was negative at the test line (T). The sensitivity of antigen detected at each test line was about equal for the three analyte control solutions, with clear detection down to about 0.4 to 0.2 ng/ml. This method shows that a single dipstick test can detect and identify the presence of P. falciparum, P. vivax 210 or 247 antigen in a single sample and at high levels of sensitivity.

### Example 7

### Assays for the Detection of Dengue Viruses

Combined Dengue 1-4 and Flavivirus Dipstick Assay

A panel assay was developed for detection of the genus Flavivirus in combination with detection of any of Dengue virus species 1-4 on the same dipstick. Three lines were printed on each dipstick including a control line and a test line for each capture antibody, the one proximal to the control made with monoclonal antibody 4G2 (flavivirus specific) and the one distal to the control made with monoclonal antibody 2H2 (Dengue 1-4 specific). Both capture antibodies were printed at 2 mg/ml. Dipsticks were prepared using both monoclonal antibodies labeled with colloidal gold and applied together in the conjugate pad.

The dipsticks were tested against analyte control solutions for Dengue 2 including 10×, 100×, 500×1000×, 2000× dilutions of Dengue 2 inactivated virus particles (Microbix Biosystems Inc., Ontario Canada) in PBS with 0.1% Tween-20 detergent. Also, a control solution containing PBS and Tween-20 was used. The assay was otherwise performed essentially as described above for the Pf2A10* Gold Assay.

The results are shown in FIG. 1, panel B. All tests developed a control line and the buffer-detergent tested dipstick was negative at the test line. The test lines for the analyte control solutions showed detection of Dengue 2 antigen down to about a 1:2,000 dilution of the antigen and this was equally detected by either the flavivirus capture reagent or the Dengue 1-4 capture reagent.

### Example 8

### Assays for the Detection of Encephalitis Viruses

This example discloses single and multiple analyte assays for detection of several encephalitis viruses.

A. Single Analyte Encephalitis Assays

1. St. Louis Encephalitis (SLE) Dipstick Assay:

In this assay, dipsticks were prepared with purified 6B6C-1 monoclonal antibody as capture and colloidal gold labeled monoclonal antibody 4A4C-4 or 6B6C-1, applied to the conjugate pad.

The dipsticks were tested against control analyte solutions for SLE Strain TBH-28 with 10×, 100×, 500×1,000×, or 2,000× dilutions of SLE Strain TBH-28 in PBS with 0.1% Tween-0 or 0.5% NP-40. A control solution containing PBS with either detergent also was used. The assay was otherwise performed essentially as described above for the Pf2A10* Gold Assay.

All tests developed a control line and the buffer-detergent tested dipsticks were negative at the test line. The test line for the analyte solutions where antibody 6B6C-1 was the capture and antibody 4A4C-4 was the conjugate showed the best sensitivity, down to about 1:2,000 dilution.

2. Western Equine Encephalitis (WEE) Dipstick Assay:

In this assay, dipsticks were prepared with purified 2A3D-5 monoclonal antibody immobilized as capture and colloidal gold labeled monoclonal antibody 2B1C-6 or 2A3D-5, applied to the conjugate pad.

The dipsticks were tested against control analyte solutions for WEE Strain Fleming with 10×, 100×, 500×1,000×, or 2,000× dilutions of WEE Strain Fleming in PBS with 0.5% NP-40. A control solution with NP-40 also was used. The assay was otherwise performed essentially as described above for the Pf2A10* Gold Assay.

All tests developed a control line and the buffer-detergent tested dipstick was negative at the test line. The test line for the analyte solutions where antibody 2A3D-5 was the capture and antibody 2B1C-6 was the conjugate showed the best sensitivity, down to about 1:2,000 dilution.

3. Eastern Equine Encephalitis (EEE) Dipstick Assay:

In this assay, dipsticks were prepared with purified 1A4B-6 monoclonal antibody immobilized as capture and colloidal gold labeled monoclonal antibody 1B5C-3 or 1A4B6, applied to the conjugate pad.

The dipsticks were tested against control analyte solutions for EEE strain NJ/60 with 10×, 100×, 500×1000×, or 2000× dilutions of EEE strain NJ/60 in PBS with 0.5% NP-40. A control solution with NP-40 also was used. The assay was otherwise performed essentially as described above for the Pf2A10* Gold Assay.

All tests developed a control line and the buffer-detergent tested dipstick was negative at the test line. The test lines gave about the same sensitivity down to about 1:2,000 dilution when antibody 1A4B-6 was the capture and antibody 1B5C-C was the conjugate or a sensitivity down to about 1:1,000 dilution for antibody 1A4B-6 as both the capture and the conjugate.

B. Combined Analyte Encephalitis Assay

A panel assay was developed for simultaneous detection of SLE, WEE and EEE viruses in a single sample using a single dipstick. Four lines were printed on each dipstick including a control line and a test line for each capture antibody, the one proximal to the control made with monoclonal antibody 6B6C-1 (Flavivirus cross-reactive) the one most distal to the control made with monoclonal antibody 1B5C-3 (EEE specific) and the one intermediate in position to the control made with monoclonal antibody 2A3D-5 (WEE specific). Antibodies 4A4C-4 (SLE specific), 2B1C-6 (WEE specific) and 1A4B-6 (broad alphavirus reactive) were each conjugated to colloidal gold and were applied together in the conjugate pad.

The dipsticks were tested against control analyte solutions containing a mixture of the encephalitis viruses (SLE, WEE, and EEE) at 10×, 100×, 500×1,000×, or 2,000× dilutions in PBS with 0.5% NP-40. A control solution with NP-40 also was used. The assay was otherwise performed essentially as described above for the Pf2A10* Gold Assay.

The results are shown in FIG. 1, panel C. All tests developed a control line and the buffer-detergent tested dipstick was negative at the test line. The test lines for the control solutions showed detection of all three encephalitis viruses to approximately equal sensitivities down to a dilution of 1:2,000 of mixed viruses.

### Example 9

### Plastic Cassette for Lateral-Flow Formats

For a lateral flow immunoassay format, a plastic cassette was constructed containing a novel filter assembly, shown in FIG. 3. The filter assembly depicted in FIG. 3C (bottom view) and FIG. 3D (side view) shows a filter clip that snaps into the body of a plastic hollow cassette which contains an assay strip therein (FIGS. 3A and 2B). A filter membrane is disposed within the area bounded by the filter clip and is held directly above the wick/sample pad when the filter assembly is secured into the plastic cassette. The filter assembly is shaped such that a trough is present to hold a volume of fluid above the filter membrane (FIG. 3C, top view).

When fully assembled, sample is added to the trough in the filter assembly above the filter, and liquid passes through the filter, removing debris such as is present in mosquito/parts extracts. The liquid then contacts the wick/sample pad and moves up though the assay strip.

The filter assembly is removable and use is optional when the test solution is relatively free from debris. Lateral-flow assays, which were performed with dipsticks contained within plastic cassettes as shown in FIG. 3A, were evaluated for detection of Plasmodium analytes. The sensitivity of this format is comparable to simply inserting the dipstick without the cassette into the control solution.

### Example 10

### Lab and Field Testing of Plasmodium Infected Mosquitoes

The following antibodies were used: Pf2A10 Mab, 3.6 mg/ml, obtained from and Pv247 Mab, 3.6 mg/ml, both available from the CDE. All antibodies were desalted using 0.1 M phosphate buffer (pH 7.0). The procedure was performed on a BioRad BioLogic Workstation. Recombinant Pf+antigen (25 micrograms lyophilized), recombinant Pv210+antigen (25 micrograms lyophilized), and recombinant Pv247+antigen (25 micrograms lyophilized) were used. All antigens were obtained from the CDC and reconstituted using mosquito grinding solution (see above), which was diluted further in the same buffer for testing. As negative controls, uninfected Anopheles stephensi mosquitoes were used. For testing, lab-infected mosquitoes infected with Pf, Pv210, and Pv247 were used. In addition, light trap-captured and human bait-collected mosquito specimens were collected from field studies in Kenya, Peru, Indonesia, and Thailand.

Selected monoclonal antibodies were conjugated with colloidal gold and employed in the preparation of dipsticks, both singly and in combination to form a panel. Each stick was prepared with its own internal positive control to indicate reagent presence and wicking ability. Recombinant antigen preparations and laboratory-infected mosquitoes were assayed by circumsporozoite (“CS”) ELISA to determine antigen concentration and corresponding sporozoite estimation of the sample. An aliquot of the same sample was subjected to assay with dipsticks (following the steps shown in FIG. 2) to determine wicking assay sensitivity and concordance with its CS ELISA value (see Wirtz et al. supra for CS ELISA details).

The field specimens of mosquitoes from Kenya, Thailand and Peru were assayed by CS ELISA to determine antigen concentration and relative sporozoite load. An aliquot of the same sample was subjected to assay with dipsticks (following the steps shown in FIG. 2) to determine wicking assay sensitivity and concordance with the CS ELISA value.

Results of Individual Test Dipsticks

Lab and field evaluations of Pf, Pv210, and Pv247 individual dipsticks (6 mm wide) demonstrated the following overall sensitivities and correlation to the CS ELISA:

Pf: detection to 212 pg of equivalent CS protein, equivalent to about 800 sporozoites;

Pv210: detection to 56 pg of equivalent CS protein, equivalent to about 500 sporozoites;

Pv247: detection to 2500 pg which is equivalent to about 300 sporozoites.

When compared to CS ELISA, there were no false positives. The sensitivity and specificity of both dipstick tests was between 90 and 100%.

Panel Assays

Lab and field evaluations of panel dipsticks (4 mm wide) demonstrated the following overall sensitivity and correlation to the CS ELISA:

Pf: detection to 60 pg of equivalent CS protein, equivalent to about 225 sporozoites;

Pv210: detection to 60 pg of equivalent CS protein, equivalent to about 325 sporozoites;

Pv247: detection to 160 pg which is equivalent to about 25 sporozoites.

There was a 100% positive predictive value and correlation between CS ELISA and dipstick results. Some batches showed limited false positives (<5%) in Pv210 and Pv247 detection zones. See FIGS. 4, 5 and 6 for sensitivity curves of each detection zone. Representative sample results with mosquitoes are shown in FIG. 7.

While the invention has been described in detail and with reference to specific embodiments thereof, it will be apparent to one skilled in the art that various changes and modifications can be made therein without departing from the spirit and scope of the invention.

All references cited above are incorporated by reference herein to the same extent as if they were individually incorporated by reference.

What is claimed is:

1. A method for analyzing an arthropod sample for the presence of one or more analytes associated with the pathogen that causes human malaria, comprising:
a) contacting a liquid permeable support with the arthropod sample and one or more detectable analyte-specific reagents that bind specifically to a protein analyte associated with a Plasmodium sporozoite, if present, to form analyte-reagent complexes, said support comprising at least one detection area, said area having an analyte-specific capture reagent immobilized therein, said capture reagent specific for the protein analyte associated with the Plasmodium sporozoite, said capture reagent being adapted for capturing the analyte-reagent complexes; and
b) detecting the presence of the detectable analyte-specific reagent in the detection area, indicating the presence of the analyte in the sample.

2. The method of claim 1, wherein the detectable analyte-specific reagent further comprises a detectable moiety selected from the group consisting of a colored moiety, a magnetic moiety, a radioactive moiety and an enzyme.

3. The method of claim 1, wherein the detectable analyte-specific reagent is deposited on the support prior to contacting the sample.

4. The method of claim 1, wherein at least three detectable analyte-specific reagents for at least three different arthropod-carried agents associated with human malaria are employed and the support comprises at least three capture reagents immobilized onto at least three different detection areas.

5. The method of claim 1, wherein the arthropod is a mosquito.

6. The method of claim 5, wherein the sample is homogenized with a grinding solution prior to contact with said support.

7. The method of claim 1, wherein the support further comprises a control area having immobilized therein at least one reagent suitable for capturing the detectable analyte-specific reagent.

8. The method of claim 1, further employing at least two detectable analyte-specific reagents, said regents specific for a protein associated with Plasmodium falciparum circumsporozoite and a second specific for a protein associated with a Plasmodium vivax sporozoite and at least two different detection areas, one area having immobilized therein a capture reagent specific for the protein associated with Plasmodium falciparum sporozoite, and a second area having immobilized therein a capture reagent specific for the protein associated with the Plasmodium vivax sporozoite.

9. The method of claim 8, wherein the Plasmodium vivax sporozoite is Plasmodium vivax 210.

10. The method of claim 8, wherein the Plasmodium vivax sporozoite is Plasmodium vivax: 247.

11. The method of claim 1, wherein the analyte-specific reagents are monoclonal antibodies.

12. The method of claim 1, wherein the detectable analyte-specific reagents are gold-antibody conjugates.

13. The method of claim 1, wherein the detectable analyte-specific reagents are colored latex-antibody conjugates.

14. A method for analyzing an arthropod sample for the presence of at least one analyte associated with at least one type of arthropod-carried agent, wherein the arthropod-carried agent is a togavirus, comprising:
a) contacting a liquid permeable support with the arthropod sample and a detectable analyte-specific reagent that binds to an analyte associated with the togavirus, if present, to form analyte-reagent complex, said support comprising a detection area, said area having an analyte-specific capture reagent immobilized therein, said capture reagent specific for the analyte associated with the togavirus, said capture reagent being adapted for capturing the analyte-reagent complex; and
b) detecting the presence of the detectable analyte-specific reagent in the detection area, indicating the presence of the analyte in the sample.

15. The method of claim 14, wherein the togavirus is an encephalitis virus.

16. The method of claim 14, wherein the togavirus is a flavivirus.

17. The method of claim 16, wherein the flavivirus is Dengue.

18. The method of claim 16, wherein the flavivirus is an encephalitis virus.

19. The method of claim 14, wherein the detectable analyte-specific reagent further comprises a detectable moiety selected from the group consisting of a colored moiety, a magnetic moiety, a radioactive moiety and an enzyme.

20. The method of claim 14, wherein the detectable analyte-specific reagent is deposited on the support prior to contacting the sample.

21. The method of claim 14, wherein three detectable analyte-specific reagents are used to detect three different encehpalitis causing viruses and the support comprises three capture reagents immobilized onto three different detection areas.

22. The method of claim 14, wherein the arthropod is a mosquito.

23. The method of claim 14, wherein the sample is homogenized with a grinding solution prior to contact with said support.

24. The method of claim 14, wherein the support further comprises a control area having immobilized therein at least one reagent suitable for capturing the detectable analyte-specific reagent.

25. The method of claim 21, wherein said three viruses are Saint Louis Encephalitis virus, Western Equine encephalitis virus and Eastern Equine encephalitis virus.

26. The method of claim 14, wherein the analyte specific reagents are monoclonal antibodies.

27. The method of claim 14, wherein the detectable analyte-specific reagents are gold-antibody conjugates.

28. The method of claim 14, wherein the detectable analyte-specific reagents are colored latex-antibody conjugates.

29. A method for analyzing an arthropod sample for the presence of an analyte associated with a Ross River virus arthropod-carried agent, comprising:
a) contacting a liquid permeable support with the arthropod sample and a detectable analyte-specific reagent that binds to an analyte associated with Ross River virus, if present, to form analyte-reagent complex, said support comprising a detection area, said area having an analyte-specific capture reagent immobilized therein, said capture reagent specific for the analyte associated with Ross River virus, said capture reagent being adapted for capturing the analyte-reagent complex; and
b) detecting the presence of the detectable analyte-specific reagent in the detection area, indicating the presence of the analyte in the sample.

30. A method for analyzing an arthropod sample for the presence of two or more analytes associated with an arthropod-carried agent, comprising:
a) contacting a liquid permeable support with the arthropod sample and at least two detectable analyte-specific reagents that bind to each of the analytes, if present, to form analyte-reagent complexes, said support comprising at least two detection areas, said areas each having an analyte-specific capture reagent immobilized therein, said capture reagent being adapted for capturing one of the analyte-reagent complexes; and
b) detecting the presence of the detectable analyte-specific reagent in each of the detection areas, indicating the presence of the analyte in the sample.

31. A kit for analyzing an arthropod sample for the presence or absence of at least one analyte associated with an arthropod-borne agent, comprising a liquid permeable support for contacting with said arthropod sample and at least one detectable analyte-specific reagent that forms an analyte-reagent complex with said analyte, said support comprising at least two detection areas having a capture reagent immobilized therein, said capture reagent being adapted for capturing the analyte-reagent complex.

32. The kit of claim 31, further comprising at least two detectable analyte-specific reagents for at least two different arthropod-associated agents, and wherein the support further comprises at least two capture reagents immobilized onto at least two different detection areas.

33. The kit of claim 31, further comprising at least three detectable analyte-specific reagents for at least three different arthropod-associated agents, and wherein the support further comprises at least three capture reagents immobilized onto at least three different detection areas.

34. The kit of claim 31, wherein the kit is adapted for analyzing a sample suspected of containing mosquitoes.

35. The kit of claim 31, further comprising a grinding solution for homogenizing said sample.

36. The kit of claim 31, wherein the support further comprises a control area having immobilized therein at least one analyte for capturing uncomplexed detectable analyte-specific reagent.

37. The kit of claim 31, further comprising at least two detectable analyte-specific reagents, said regents specific for a protein associated with Plasmodium falciparum sporozoite and a second specific for a protein associated with a Plasmodium vivax sporozoite and at least two different detection areas, one area having immobilized therein a capture reagent specific for the protein associated with Plasmodium falciparum sporozoite, and a second area having immobilized therein a capture reagent specific for the protein associated with the Plasmodium vivax sporozoite.

38. The kit of claim 31, wherein the analyte-specific reagents are monoclonal antibodies.

39. The kit of claim 31, wherein the detectable analyte-specific reagents are gold-antibody conjugates.

40. The kit of claim 31, wherein the detectable analyte-specific reagents are colored latex-antibody conjugates.

41. The kit of claim 31, wherein the support further comprises at least one detectable analyte-specific reagent for an analyte associated with a togavirus and at least one detection area having immobilized therein a capture reagent specific for an analyte associated with the togavirus.

42. The kit of claim 31, further comprising a hollow plastic cassette for holding the liquid permeable support.

43. The kit of claim 42, wherein the plastic cassette is formed with an opening for receiving a filter assembly adapted to clip onto the cassette above the liquid permeable support, the kit further comprising the filter assembly with a filter membrane disposed therein for filtering the sample prior to contacting the support.
